(19) United States
(12) Patent Application Publication
(10) Pub. No.: US 2021/0154193 A1
(43) Pub. Date: May 27, 2021

(54) PHARMACEUTICAL COMPOSITIONS FOR THE TREATMENT OF PULMONARY HYPERTENSION

(71) Applicant: PHPrecisionMed, LLC, Skokie, IL (US)

(72) Inventors: Anna R. Hemnes, Nashville, TN (US); Stuart Rich, Skokie, IL (US)

(21) Appl. No.: 17/101,334

(22) Filed: Nov. 23, 2020

Related U.S. Application Data
(60) Provisional application No. 62/939,834, filed on Nov. 25, 2019.

(51) Int. Cl. A61K 31/506; A61K 9/48

(52) U.S. Cl. CPC A61K 31/506; A61K 9/4866; A61K 9/4816; A61K 9/4891

(57) ABSTRACT

The present disclosure pertains to the use of Imatinib or a pharmaceutically acceptable salt thereof for the treatment of pulmonary hypertension. A patient status relative to their baseline status of at least one of a proteomic biomarker, a protein biomarker, a transcriptomic biomarker, a patient specific genetic signature, an RNA expression biomarker signature, or a timed walk distance may be used in adjusting at least one of a dosage of imatinib or a treatment duration.

### CLAIM TO PRIORITY

This application claims the benefit of the following provisional application, each of which is hereby incorporated by reference in its entirety: U.S. Application No. 62/939,834, filed Nov. 25, 2019 (PHPM-0001-P01).

### BACKGROUND

### Field

The disclosure is in the field of treatment of pulmonary hypertension, including pulmonary arterial hypertension (PAH).

### Description of the Related Art

Pulmonary hypertension (PH) is a disease in which the pressure within the pulmonary arterial circulation becomes elevated. This can occur in isolation without a known cause, or in association with of a number of cardiac and pulmonary conditions. When it occurs, it causes a worsening of the condition resulting in severe debilitation, right heart failure and death. The World Health Organization has classified pulmonary hypertension five main categories. They are:

Group 1: this group includes pulmonary arterial hypertension (PAH), including idiopathic pulmonary arterial hypertension (with no clear cause), inherited pulmonary arterial hypertension (linked to genes inherited in families), pulmonary arterial hypertension resulting from congenital heart disease, liver disease, HIV and connective tissue disease, such as scleroderma and systemic lupus. Pulmonary arterial hypertension [pulmonary arterial hypertension], is a life-threatening orphan disease affecting less than 10% of pulmonary hypertension patients and characterized by a marked and sustained elevation of pulmonary artery pressure. Current therapeutic approaches for the treatment of chronic pulmonary arterial hypertension mainly provide symptomatic relief, as well as some improvement of prognosis.

Group 2: pulmonary hypertension resulting from left heart disease. This is a common form of pulmonary hypertension and affects approximately half of pulmonary hypertension sufferers.

Group 3: pulmonary hypertension due to chronic lung disease including COPD and emphysema, and interstitial lung diseases. It affects approximately half of pulmonary hypertension sufferers.

Group 4: pulmonary hypertension due to chronic blood clots in lungs and affects approximately 20% of pulmonary hypertension sufferers.

Group 5: pulmonary hypertension due to a variety of causes.

All of the currently approved therapies are for Group I type pulmonary hypertension. They help relieve symptoms to improve quality of life and may slow down progression of the disease. They are therapies that manage pulmonary arterial hypertension. These therapeutic approaches have been with pulmonary vasodilators (PV). Pulmonary vasodilators do not affect the underlying morphological changes of the small pulmonary arteries. Data has shown that pulmonary arterial hypertension is largely a vascular proliferative disease, and as a result these therapies are only partially effective in providing symptomatic relief and in delaying disease progression.

No therapy has ever been shown to halt the progression of the disease or induce regression of the disease. The instant disclosure is a response to that need.

### SUMMARY

The disclosure relates to a method of use of 4-(4-methylpiperazin-1-ylmethyl)-N-[4-methyl-3-(4-pyridin-3-yl)pyrimidin-2-ylamino)phenyl]-benzamide (also known as “imatinib” or a pharmaceutically acceptable salt thereof for the treatment of pulmonary hypertension.

Imatinib, sold under the brand names GLEEVEC™ among others, is a medication used to treat cancer. Specifically, it is used for chronic myelogenous leukemia (CIVIL) and acute lymphocytic leukemia (ALL) that are Philadelphia chromosome-positive (Ph⁺), certain types of gastrointestinal stromal tumors (GIST), hypereosinophilic syndrome (HES), chronic eosinophilic leukemia (CEL), systemic macrocytosis, and myelodysplastic syndrome.

In an embodiment, the present disclosure provides methods of treating pulmonary hypertension in a patient comprising administering Imatinib or a pharmaceutically acceptable salt thereof to the patient according to a dosing regimen, which comprises modulating doses of Imatinib or a pharmaceutically acceptable salt thereof over an interval to the patient until the effective dosage is achieved.

In certain embodiments, the modulating dosing regimen contemplates a linear increase in Imatinib or a pharmaceutically acceptable salt thereof dosage. In other embodiments, the modulating dosage regimen includes a stepwise increase in Imatinib or a pharmaceutically acceptable salt thereof dosage.

In certain embodiments, the effective dose is the dose at which an improved measurable response outcome is observed. This dose may include from about 100 mg/day to about 500 mg/day, about 120 mg/day, about 240 mg/day, about 360 mg/day, about 480 mg/day, or about 120 mg once, twice, three or four times per day.

In a certain embodiment, the modulated dose in the dosing regimen is administered over an interval of about 2 weeks to about 16 weeks.

In another embodiment, the present disclosure is directed to a method of improving the measurable response outcome in a patient suffering from pulmonary hypertension, comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) measuring the patient's measurable response outcome; (c) administering Imatinib or a pharmaceutically acceptable salt thereof at a dose of about 120 mg/day; (d) monitoring the patient for an improved measurable response outcome after an interval of at least 2 weeks after dosing in step (c); (e) modulating the dose of Imatinib or a pharmaceutically acceptable salt thereof when the measurable response outcome is not improved; (f) repeating steps (d)-(e) for a maximum of about 16 weeks until the measurable response outcome is improved; and optionally (g) administering a maintenance dose of Imatinib or a pharmaceutically acceptable salt thereof to the patient when the patient achieves an improved measurable response outcome.

In a certain embodiment, the interval in the dosing regimen includes at least 2 weeks, at least 3 weeks, or at least 4 weeks.

In certain embodiments, the modulating dose during the dosing regimen includes about 120 mg/day, about 240 mg/day, about 360 mg/day or about 480 mg/day or 120 mg administered once, twice, three, or four times as day.

In certain embodiments, the improved measurable response may include a timed walk distance, such as a 6-minute walk distance or other walk distance of a specified duration, measure that includes a distance of greater than about 6 meters, greater than about 10 meters, greater than about 15 meters, greater than about 20 meters, greater than about 25 meters, greater than about 30 meters, greater than about 35 meters, greater than about 40 meters and greater than about 45 meters compared to the six minute walk distance measured distance pre-treatment, pre-dosing or on a chronic or maintenance dose.

In certain embodiments, the measurable response outcomes include significant improvement or significantly improved in a 6-minute distance measure of greater than about 35 meters, greater than about 40 meters and greater than about 45 meters compared to the measured distance pre-treatment pre-dosing or on chronic or maintenance dosing. In other embodiments, the measurable response outcomes include exceptional improvement or exceptionally improved in a 6-minute walk distance measure of greater than about 45 meters compared to the measured distance pre-treatment pre-dosing or on a chronic or maintenance dose.

In another embodiment, the present disclosure is directed to a method of treating pulmonary hypertension in a patient suffering therefrom, said method comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) measuring the patient's measurable response outcome; (c) administering Imatinib or a pharmaceutically acceptable salt thereof at a dose of about 120 mg/day; (d) monitoring the patient for an improved measurable response outcome after an interval of at least 2 weeks after dosing in step (c); (e) modulating the dose of Imatinib or a pharmaceutically acceptable salt thereof when on the measurable response outcome is not improved; (f) repeating steps (d)-(e) for a maximum of 16 weeks or until the measure response outcome is improved; and (g) administering the effective dose of Imatinib or a pharmaceutically acceptable salt thereof to the patient when the patient achieves improved measurable response outcome to treat pulmonary hypertension.

In another embodiment, an acid resistant capsule includes (a) a capsule comprising at least one enteric polymer; and (b) a therapeutically effective amount of Imatinib or a pharmaceutically acceptable salt thereof.

In another embodiment, a pharmaceutical composition includes an acid resistant capsule comprising (a) a capsule comprising at least one enteric polymer; and (b) a therapeutically effective amount of Imatinib or a pharmaceutically acceptable salt thereof; and (c) one or more pharmaceutically acceptable carrier(s). In certain embodiments, the enteric polymer has a degree of ionization less than about 15%, preferably less than 12%, preferably less than 10%, or more preferably from 0.1% to 9%.

In another embodiment, a method of treating a human subject having pulmonary hypertension may include: (a) a dosing regimen comprising: (i) administering an initial dose of Imatinib or a pharmaceutically acceptable salt thereof to the human subject; (ii) administering successively higher doses of Imatinib or a pharmaceutically acceptable salt thereof and monitoring the subject for a measurable response outcome; and (c) administering an effective dose to the human subject to treat pulmonary hypertension, wherein the effective dose is the dose that achieves an improved measurable response outcome in the subject.

In certain embodiments, any Imatinib or pharmaceutically acceptable salt thereof dose, an initial dose, successively higher dose(s), or effective dose of Imatinib or a pharmaceutically acceptable salt thereof includes a pharmaceutical composition comprising an acid resistant capsule comprising (a) a capsule comprising at least one enteric polymer; and (b) a therapeutically effective amount of imatinib or a pharmaceutically acceptable salt thereof; and optionally (c) one or more pharmaceutically acceptable carrier(s).

In certain embodiments, an initial dose of Imatinib or a pharmaceutically acceptable salt thereof includes doses from about 100 mg/day to about 500 mg/day, about 120 mg/day, about 240 mg/day, about 360 mg/day, about 480 mg/day, or about 120 mg once, twice, three or four times per day.

In certain embodiments, the effective dose may be determined by a dosing regimen, such as that described herein. Additionally, the effective dose may include the dose at which an improved measurable response outcome is observed. This dose may include from about 100 mg/day to about 500 mg/day, about 120 mg/day, about 240 mg/day, about 360 mg/day, about 480 mg/day, or about 120 mg once, twice, three or four times per day.

In certain embodiments, a successively higher dose of Imatinib or a pharmaceutically acceptable salt thereof, may be about 120 mg/day, about 240 mg/day, about 360 mg/day and about 480 mg/day.

In another embodiment, the present disclosure is directed to methods of identifying patient specific biomarkers for patients with pulmonary hypertension that are responsive to Imatinib or a pharmaceutically acceptable salt thereof treatment, comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) obtaining a biological sample from said patient and determining levels of said protein or transcriptomic biomarkers selected from one or more of ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, FGFb, DDR1, and DDR2; (c) administer a dose of Imatinib or a pharmaceutically acceptable salt thereof to the patient according to the dosing regimen; (d) monitor the patient for a measurable response outcome after at least 2 weeks; (e) obtain a biological sample from the patient who achieved an improved measurable response outcome and compare the biomarker levels to the sample obtained in (b); (f) identify the modulated biomarkers that are a predictor of patients responsive to Imatinib or a pharmaceutically acceptable salt thereof to treat pulmonary hypertension; and optionally (g) administer to the responsive patient an effective dose of Imatinib of a pharmaceutically acceptable salt thereof to the patient having the modulated biomarkers to treat pulmonary hypertension.

In certain embodiments, the effective dose may be determined by a dosing regimen, such as that described herein. Additionally, the effective dose includes the dose at which an improved measurable response outcome is observed. This dose may include from about 100 mg/day to about 500 mg/day, about 120 mg/day, about 240 mg/day, about 360 mg/day, about 480 mg/day, or about 120 mg once, twice, three or four times per day.

In certain embodiments, the biological sample may include blood, plasma, urine, cerebral spinal fluid, feces, cell and tissue where protein or transcript levels in the blood, urine, tissue or cells of one or more of ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, FGFb, DDR1, and DDR2 may be detected.

In another embodiment, the present disclosure provides a method of identifying a patient specific genetic and/or protein signature for patients with pulmonary hypertension that are responsive to Imatinib or a pharmaceutically acceptable salt thereof treatment, comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) obtaining a biological sample from said patient and determining a genetic signature of the patient selected from genes that encode one or more proteins selected from ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, FGFb, DDR1, and DDR2, DSG2, EPDR1, SCD5, MGAT5, RHOQ, P2RY5, ZNF652, RALGPS2, MKNL1, RAPFEF2, TPD52, PIAS, HPRT, TPD52, ADCY8, COL15A1, TGFB1, MYH13, MTFR1, ANKRD6, DNAH1, FER1L5, ZDHHC20, CDH23, CRCP, WNT5A, WIF1 and genes involved in the metabolism and down-stream signaling thereof (c) administer a dose of Imatinib or a pharmaceutically acceptable salt thereof to the patient the according to the dosing regimen; (d) monitor the patient for a measurable response outcome after at least 2 weeks; (e) obtain a biological sample from the patient who achieved the improved measurable response outcome and compare the genetic signatures to the sample obtained in (b); (f) identify the genetic signatures as predictors of patients responsive to Imatinib or a pharmaceutically acceptable salt thereof to treat pulmonary hypertension; and optionally (g) administer to the responsive patient an effective dose of Imatinib of a pharmaceutically acceptable salt thereof to the patient having the genetic signatures to treat pulmonary hypertension.

In certain embodiments, the biological sample may be provided by a patient and may include blood, plasma, urine, cerebral spinal fluid, feces, cell and tissue. In certain embodiments, any Imatinib or pharmaceutically acceptable salt thereof dose, an initial dose, successively higher dose(s), or effective dose of Imatinib or a pharmaceutically acceptable salt thereof includes a pharmaceutical composition comprising an acid resistant capsule comprising (a) a capsule comprising at least one enteric polymer; and (b) a therapeutically effective amount of imatinib or a pharmaceutically acceptable salt thereof; and optionally (c) one or more pharmaceutically acceptable carrier(s). In certain embodiments, the effective dose may be determined by a dosing regimen, such as that described herein. Additionally, the effective dose includes the dose at which an improved measurable response outcome is observed. This dose may include from about 100 mg/day to about 500 mg/day, about 120 mg/day, about 240 mg/day, about 360 mg/day, about 480 mg/day, or about 120 mg once, twice, three or four times per day.

In another embodiment, the present disclosure provides a method of treating a patient with pulmonary hypertension, the method comprising the steps of: (a) identifying the genetic signature of patients with pulmonary arterial hypertension who have an improved measurable response outcome to Imatinib or a pharmaceutically acceptable salt thereof; (b) identifying the patient at risk of developing pulmonary hypertension, diagnosed with pulmonary hypertension, patients characterized by the WHO classification groups 1, 2, 3, 4 or 5 for pulmonary hypertension, other than pulmonary arterial hypertension, have the similar genetic signature common to patients with pulmonary arterial hypertension that have an improved measurable response outcome to Imatinib or a pharmaceutically acceptable salt thereof; and (c) administering the effective dose of Imatinib or a pharmaceutically acceptable salt thereof to said patients with pulmonary hypertension.

In certain embodiments, the effective dose may be determined by a dosing regimen, such as that described herein. Additionally, the effective dose includes the dose at which an improved measurable response outcome is observed. This dose may include from about 100 mg/day to about 500 mg/day, about 120 mg/day, about 240 mg/day, about 360 mg/day, about 480 mg/day, or about 120 mg once, twice, three or four times per day.

In certain embodiments, the genetic signature may be identified by a responsive patient genetic signature method as described herein, and may include patients having one or more of the following genes selected from ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, FGFb, DDR1, and DDR2, DSG2, EPDR1, SCD5, MGAT5, RHOQ, P2RY5, ZNF652, RALGPS2, MKNL1, RAPFEF2, TPD52, PIAS, HPRT, TPD52, ADCY8, COL15A1, TGFB1, MYH13, MTFR1, ANKRD6, DNAH1, FER1L5, ZDHHC20, CDH23, CRCP, WNT5A, and WIF1.

In a further embodiment, the present disclosure provides for a method of chronic management of pulmonary hypertension in a patient in need thereof, said method comprising: (a) adjusting the effective dose, comprising: (a) monitoring the proteomic biomarker levels and/or RNA expression biomarker signature levels of responsive patients with pulmonary hypertension who show an improved measurable response outcome to treatment with Imatinib or a pharmaceutically acceptable salt thereof to periodically detect changes in the levels of the proteomic and RNA expression biomarkers levels and comparing them to prior measurable response outcome selected from a 6 minute walk distance, echocardiogram, Functional Class, Quality of Life questionnaire, and hemodynamics; (b) adjusting the Imatinib or a pharmaceutically acceptable salt thereof dose downward in patients who maintain an exceptional measurable outcome response when the biomarker levels fall significantly; (c) adjusting the Imatinib or a pharmaceutically acceptable salt thereof dose upward in patients who lose an exceptional measurable response outcome when the biomarker levels increase; and (d) administering the adjusted effective dose to treat pulmonary hypertension.

In certain embodiments of the present disclosure, a periodic detection of change of levels which includes at least every month, every two months, every 3 months, every 4 months, every 5 months, every 6 months.

In certain embodiments of the present disclosure, an exceptional measurable response outcome includes a 6-minute walk distance of an increase of more than at least 45 meters compared to pre-treatment or prior levels observed.

In certain embodiments, an exception measurable response outcome includes changes to hemodynamics which include a rise of fall in levels of certain hemodynamic parameters relative to previous treatment or dosing regimen. The hemodynamic parameters include pulmonary hemodynamic parameters, which includes changes in levels of mean pulmonary arterial pressure, mean pulmonary artery wedge pressure, systolic arterial pressure, heart rate, cardiac output, pulmonary vascular resistance, or systemic vascular resistance.

In another embodiment, the present disclosure provides a method of treating pulmonary hypertension comprising administering to a patient in need thereof an effective dose to treat pulmonary hypertension of Imatinib or a pharmaceutically acceptable salt thereof of about 120 mg/day to about 480 mg/day.

In certain embodiments, the effective dose includes from about 100 mg/day to about 500 mg/day, about 120 mg/day, about 240 mg/day, about 360 mg/day, about 480 mg/day, or about 120 mg once, twice, three or four times per day.

In certain embodiments, the responsive RNA expression biomarker genetic signature may be identified by a responsive patient genetic signature method as described in paragraph [00108], and may include patients having one or more of the following genes selected from ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, FGFb, DDR1, and DDR2, DSG2, EPDR1, SCD5, MGAT5, RHOQ, P2RY5, ZNF652, RALGPS2, MKNL1, RAPFEF2, TPD52, PIAS, HPRT, TPD52, ADCY8, COL15A1, TGFB1, MYH13, MTFR1, ANKRD6, DNAH1, FER1L5, ZDHHC20, CDH23, CRCP, WNT5A, and WIF1.

In certain embodiments, the responsive proteomic biomarkers may be identified by a response patient proteomic biomarker method as described in paragraph [00106], and may include patients having one or more of the following proteomic biomarkers ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, FGFb, DDR1, and/or DDR2.

In another embodiment, the present disclosure provides a method of treating pulmonary hypertension selected from (a) idiopathic or primary pulmonary hypertension, (b) familial hypertension, (c) pulmonary hypertension secondary to, but not limited to, connective tissue disease, congenital heart defects (shunts), pulmonary fibrosis, portal hypertension, HIV infection, sickle cell disease, drugs and toxins (e.g., anorexigens, cocaine), chronic hypoxia, chronic pulmonary obstructive disease, sleep apnea, and schistosomiasis, (d) pulmonary hypertension associated with significant venous or capillary involvement (pulmonary veno-occlusive disease, pulmonary capillary hem angiomatosis), (e) secondary pulmonary hypertension that is out of proportion to the degree of left ventricular dysfunction, and (f) persistent pulmonary hypertension in newborn babies comprising administering to a patient in need thereof a lowest effective dose to treat pulmonary hypertension of Imatinib or a pharmaceutically acceptable salt thereof of about 120 mg/day to about 480 mg/day.

In a certain embodiment, the effective dose includes about 120 mg/day, 240 mg/day, 360 mg/day and 480 mg/day, or 120 mg once, twice, three of four times per day.

In a further embodiment, the present disclosure provides a pharmaceutical composition comprising Imatinib or a pharmaceutically acceptable salt thereof at the effective dose to treat pulmonary hypertension and a pharmaceutically acceptable carrier.

In certain embodiments, any Imatinib or pharmaceutically acceptable salt thereof dose, an initial dose, successively higher dose(s), or effective dose of Imatinib or a pharmaceutically acceptable salt thereof includes a pharmaceutical composition comprising an acid resistant capsule comprising (a) a capsule comprising at least one enteric polymer; and (b) a therapeutically effective amount of imatinib or a pharmaceutically acceptable salt thereof; and optionally (c) one or more pharmaceutically acceptable carrier(s).

In a further embodiment, the present disclosure provides a pharmaceutical composition comprising Imatinib or a pharmaceutically acceptable salt thereof at the effective dose in the range of about 100 to about 500 mg/day to treat pulmonary hypertension and a pharmaceutically acceptable carrier.

In a certain embodiment, the effective dose includes about 120 mg/day, 240 mg/day, 360 mg/day and 480 mg/day, or 120 mg once, twice, three of four times per day.

In a further embodiment, Imatinib or a pharmaceutically acceptable salt thereof may be formulated into an oral controlled release formulation selected from immediate release, sustained release, delayed release, or a combination thereof.

These and other systems, methods, objects, features, and advantages of the present disclosure will be apparent to those skilled in the art from the following detailed description of the preferred embodiment and the drawings.

All documents mentioned herein are hereby incorporated in their entirety by reference. References to items in the singular should be understood to include items in the plural, and vice versa, unless explicitly stated otherwise or clear from the text. Grammatical conjunctions are intended to express any and all disjunctive and conjunctive combinations of conjoined clauses, sentences, words, and the like, unless otherwise stated or clear from the context.

### BRIEF DESCRIPTION OF THE FIGURES

The disclosure and the following detailed description of certain embodiments thereof may be understood by reference to the following figures:

FIG. 1 depicts a method of improving a 6-minute walking distance in a patient suffering from pulmonary hypertension by greater than about 35 meters.

FIG. 2 depicts a method of treating a patient with pulmonary arterial hypertension.

FIG. 3 depicts a method of treating a patient with pulmonary arterial hypertension.

FIG. 4 depicts a method of treating a patient with pulmonary arterial hypertension.

### DETAILED DESCRIPTION

The disclosure herein concerns treatment of pulmonary hypertension, particularly pulmonary arterial hypertension, using imatinib or a pharmaceutically acceptable salt thereof.

Imatinib is a tyrosine kinase inhibitor of PDGFR a and b kinases, Abl, DDR, and c-KIT, all of which may be relevant to the pathology of PAH, and may therefore be efficacious in the treatment of PAH. In prior clinical trials, imatinib was not shown to be successful in the treatment of PAH largely due to tolerability issues with the composition and study discontinuation (i.e. dropouts)(GHOFRANI, et al., “Imatinib in Pulmonary Arterial Hypertension Patients with Inadequate Response to Established Therapy”, AMERICAN JOURNAL OF RESPIRATORY AND CRITICAL CARE MEDICINE, Nov. 1, 2010, pp. 1171-7; HOEPER, M D, et al., “Imatinib Mesylate as Add-on Therapy for Pulmonary Arterial Hypertension”, Imatinib in Pulmonary Arterial Hypertension, Mar. 12, 2013, pp. 1128-38). On the heels of the failed clinical trial, the inventors discovered a new treatment protocol and a new enteric formulation that solved the tolerability issues and enables a demonstration of efficaciousness. The inventors have devised a method for dose escalation. Patients are tested via 6 MWD, hemodynamics, and/or other functional characterization then are administered an initial dose. After a set time, the tests are repeated to determine if there is an improvement, for example, an improvement of 30 or more meters in the 6 MWD. If there is no improvement, the dose may be escalated until an improvement is observed. If there is still no improvement, the patient discontinues treatment.

For patients who exhibit improvement, either who have gone through dose escalation or not, monitoring may be continued through continued treatment via a clinical endpoint, such as a proteomic profile or circulating biomarkers. Some key ones are PDGF, BCR-ab1, DDR, and C-Kit, which are associated with the mechanism of action pathways for imatinib. The proteomic profile or circulating biomarkers associated with responsiveness are monitored for changes, such as in levels or expression patterns. If they continue to change in the direction they changed upon improvement, then a dose reduction may be considered. Dose may continue to be reduced until the biomarkers no longer change. If the biomarkers change in a direction they were before successful treatment, then a dose escalation may be considered.

With enough accumulated data on the responsiveness of patients to imatinib and their associated genetic profile (e.g. particular mutations in key genes) and/or their proteomic profile (e.g. expression levels of particular proteins), predictions may be made for new patients. If a genetic signature or a proteomic profile of a new patient matches that of a responsive patient, imatinib may be recommended for treatment. Further, the responsive patient's effective dose, such as after dose escalation or not, may be recommended.

As used herein “measurable response” means an objective indicia of a positive response to the treatment as exhibited by the 6-minute walk test (6 MWD). A measurable response may include a moderate response or an exceptional response.

As used herein “exceptional response” means a 6 MWD of 45 meters or greater.

As used herein “moderate response” means a 6 MWD of less than 45 meters.

As used herein “tolerate” means an absence of serious adverse event.

As used herein “severe adverse event” (SAE) is a grade III or IV adverse event as defined by the National Cancer Institute (NCI). A grade III event is defined as severe and a grade IV event is generally defined as life-threatening or disabling.

As used herein “adverse event” (AE) means an unfavorable or generally unintended, even undesirable, sign, symptom, or disease associated with the use of a medical treatment. Most AE's are temporary and reverse upon withdrawal or reduction in dose of medical treatment, or with treatment of an AE.

Adverse events associated with pulmonary hypertension are, among others, nausea, edema peripheral, diarrhea, vomiting, periorbital edema, headache, dyspnea, nasopharyngitis, hypokalemia, anemia, cough, fatigue, face edema, muscle spasms, abdominal distension, blood creatinine increased, dizziness, oropharyngeal pain, rash, dyspepsia, epistaxis, alopecia, pyrexia, abdominal pain, nasal congestion, pain in extremity, upper respiratory tract infection, palpitations, urinary tract infection, non-cardiac chest pain, pruritus, respiratory tract infection, abdominal pain upper, sinusitis, and syncope.

As used herein, “escalating dosage regimen” or “dosage escalation regimen” or “escalating dosage strategy” or “dosage escalation strategy” means a dosing regimen administered to a patient wherein dosages in a series can be increased in a stepwise (subsequent dosages are greater than or equal to the immediately preceding dosage, but at least increase in dosage occur over the length of the regimen) or linear fashion (i.e., each subsequent dosage is greater than its immediately preceding dosages.

As used herein, “effective dose” means a dose that results in measurable response in the measurable response test and therefore effective to treat the disease being treated. This dose may be in the range of about 100 mg/day to about 500 mg/day, preferably about 120 mg/day to about 480 mg/day, most preferably selected from about 120 mg/day, 240 mg/day or 120 mg twice a day, 360 mg/day or 120 mg three times per day, or 480 mg/day or 120 mg four times a day. The effective dose may also be selected from about 120 mg once, twice, three or four times per day.

As used herein “proteomic biomarkers” will include biomarkers for patients suffering from pulmonary hypertension and respond treatment with Imatinib or a pharmaceutically acceptable salt thereof. These proteomic and transcriptomic biomarkers include but are not limited to, ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, TGFb, DDR1, or DDR2.

As used herein “RNA expression biomarker signature” means expression of RNA in blood, urine, tissue or cells. There may be one or more RNAs whose expression level correlates with clinical response. The transcriptomic biomarkers include but are not limited to, ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, TGFb, DDR1, DDR2.

The term “enteric polymer” as used herein and in the claims includes both a single species of polymer and mixtures of one or more enteric polymers.

The term “acid resistant” as used herein and in the claims include materials that are water insoluble under gastric conditions (conventionally simulated by pH 1.2) and readily water soluble under intestinal conditions (conventionally simulated by a pH of 6.8).

The term “degree of ionization” as used herein means the percentage of the acid groups of the enteric polymer (e.g. the succinic acid groups present in the HPMCAS polymer) that are in the ionized state (e.g., neutralized with an alkaline material (such as a base), or not in a protonated state). This parameter is calculated as degree of ionization=100/(1+10 pKa−pH) where the pKa refers to the negative logarithm of the acid ionization constant for the carboxylic acid groups on an enteric polymer, and pH refers to the negative logarithm of the proton or hydronium concentration in solution as measured using a calibrated pH electrode.

The term “hard shell” or “hard capsule shell” refers to a shell that is capable of maintaining a shape so as to be filled with and encapsulate a medicament using conventional capsule filling equipment.

The term “patient” or “subject” are used interchangeably in this specification and refer to mammals such as human patients and non-human patients, as well as experimental animals such as rabbits, rats, and mice and other animals. Animals include all vertebrates, e.g., mammals and non-mammals, such as sheep, dogs, cows, chickens, amphibians, etc.

The term “treating” includes the administration of Imatinib or a pharmaceutically acceptable salt thereof of the present disclosure to prevent or delay the onset of the symptoms, complications, or biochemical indicia of a disease, alleviating the symptoms or arresting or inhibiting further development of the diseases, condition or disorder. Treatment may be prophylactic or therapeutic suppression or alleviation of symptoms after the manifestation of the disease.

The term “about” or “approximately” means within an acceptable range for the particular parameter specified as determined by one of ordinary skill in the art, which will depend in part on how the value is measured or determined, e.g., the limitations of the measurement system. For example, “about” can mean a range of up to 20% of a given value. Alternatively, particularly with respect to biological systems or processes, the term can mean within an order of magnitude within 5-fold and more preferably within 2-fold of a value.

The term “treatment” as used herein means curative treatment and prophylactic treatment.

The term “curative” as used herein means efficacy in treating ongoing episodes of pulmonary hypertension, especially pulmonary arterial hypertension.

The term “prophylactic” means the prevention of the onset or recurrence of pulmonary hypertension, especially pulmonary arterial hypertension.

Throughout this specification and in the claims that follow, unless the context requires otherwise, the word “comprise”, or variations such as “comprises” or “comprising”, will be understood to imply the inclusion of a stated integer or step or group of integers or steps but not the exclusion of any other integer or step or group of integers or steps.

The present disclosure is directed to the treatment of pulmonary hypertension of all Groups, in particular pulmonary arterial hypertension. Examples of pulmonary hypertension that can be treated according to the disclosure include (a) idiopathic or primary pulmonary hypertension, (b) familial hypertension, (c) pulmonary hypertension secondary to, but not limited to, connective tissue disease, congenital heart defects (shunts), pulmonary fibrosis, portal hypertension, HIV infection, sickle cell disease, drugs and toxins (e.g., anorexigens, cocaine), chronic hypoxia, chronic pulmonary obstructive disease, sleep apnea, and schistosomiasis, (d) pulmonary hypertension associated with significant venous or capillary involvement (pulmonary veno-occlusive disease, pulmonary capillary hemangiomatosis), (e) secondary pulmonary hypertension that is out of proportion to the degree of left ventricular dysfunction, (f) persistent pulmonary hypertension in newborn babies.

The present disclosure is directed to the treatment of pulmonary hypertension of all Groups, in particular pulmonary arterial hypertension with imatinib or a pharmaceutically acceptable salt thereof. The preparation of Imatinib or its pharmaceutically acceptable salt thereof and the use thereof, especially as an anti-tumor agent, are described in Example 21 of U.S. Pat. No. 5,521,184.

Pharmaceutically acceptable salts of Imatinib are pharmaceutically acceptable acid addition salts, such as for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxy maleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic acid, or aromatic sulfonic acids, for example benzene-, p-toluene- or naphthalene-2-sulfonic acid. The aforementioned list is illustrative and not intended to be exhaustive.

The monomethanesulfonic acid addition salt of Imatinib (hereinafter “Imatinib mesylate” or “imatinib mesylate” or “Imatinib monomethanesulfonate”) and a preferred crystal form thereof, e.g. the beta.-crystal form, are described in PCT patent application WO99/03854 published on Jan. 28, 1999 and US equivalent, U.S. Pat. No. 6,894,051 thereof.

Imatinib or a pharmaceutically acceptable salt thereof can be administered by any route including orally, parenterally, e.g., intraperitoneally, intravenously, intramuscularly, subcutaneously, intratumorally, vaginally, rectally, or enterally, as a single daily dose or divided into multiple doses.

According to the methods and compositions of the present disclosure, Imatinib or a pharmaceutically acceptable salt thereof can be administered in combination with one or more other therapeutic agents, or sequentially or in conjunction with other therapeutic agents. In certain embodiments, (a) Imatinib or a pharmaceutically acceptable salt thereof may be used in combination with (b) at least one compound selected from compounds indicated for the treatment of pulmonary arterial hypertension, such as calcium channel antagonists, e.g. nifedipine, e.g. 120 to 240 mg/d, or diltiazem, e.g. 540 to 900 mg/d, prostacyclin, the prostacyclin analogues iloprost, flolan and treprostinil, adenosine, inhaled nitric oxide, e.g. warfarin, digoxin, endothelin receptor blockers, e.g. bosentan, phosphodiesterease inhibitors, e.g. sildenafil, norepinephrine, angiotensin-converting enzyme inhibitors e.g. enalapril or diuretics, ambrisentan; tadalafil; bosentan; treprostinil (intravenous, subcutaneous, inhaled, oral); macitentan; epoprostenol; iloprost; riociguat; selexipag anticoagulants; a combination comprising (a) and (b) as defined above and optionally at least one pharmaceutically acceptable carrier for simultaneous, separate or sequential use, in particular for the treatment of pulmonary hypertension, particularly pulmonary arterial hypertension; a pharmaceutical composition comprising such a combination; the use of such a combination for the preparation of a medicament for the delay of progression or treatment of pulmonary hypertension, particularly pulmonary arterial hypertension; and to a commercial package or product comprising such a combination.

The disclosure encompasses pharmaceutical compositions comprising Imatinib or a pharmaceutically acceptable salt thereof formulated together with a pharmaceutically acceptable carrier for use in a dosage escalation regimen.

Pharmaceutically acceptable carriers include solvents, dispersion media, coatings, antibacterial and antifungal agents (e.g., paraben, chlorobutanol, phenol sorbic acid, and the like), isotonic and absorption delaying agents, and the like that are physiologically compatible. The carrier can be suitable for intravenous, intramuscular, subcutaneous, parenteral, spinal or epidermal administration (e.g., by injection or infusion). Depending on the route of administration, the active compound, i.e., antibody, bispecific and multi-specific molecule, may be coated in a material to protect the compound from the action of acids and other natural conditions that may inactivate the compound.

Pharmaceutically acceptable carriers also include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the disclosure is contemplated. For example, the compound may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes. Supplementary active compounds can also be incorporated into the compositions.

Therapeutic compositions that are administered intravenously typically must be sterile, substantially isotonic, and stable under the conditions of manufacture and storage. The composition may be formulated as a solution, microemulsion, liposome, or other ordered structure suitable to high drug concentration. The carrier may be a solvent or dispersion medium containing, for example, water, ethanol, polyol (for example, glycerol, propylene glycol, and liquid polyethylene glycol, and the like), and suitable mixtures thereof, vegetable oils, such as olive oil, and injectable organic esters, such as ethyl oleate. The proper fluidity can be maintained, for example, by the use of a coating such as lecithin, by the maintenance of the required particle size in the case of dispersion and by the use of surfactants. In many cases, it is preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. These compositions may also contain adjuvants such as preservatives, wetting agents, emulsifying agents and dispersing agents.

Sterile injectable solutions can be prepared by incorporating the active compound in the required amount in an appropriate solvent with one or more of a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

A “pharmaceutically acceptable salt” refers to a salt that retains the desired biological activity of the parent compound and does not impart any undesired toxicological effects. Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A composition for use in a dosing regimen according to the present disclosure can be administered by a variety of methods known in the art. The route and/or mode of administration may vary depending upon the desired results. The active compounds can be prepared with carriers that protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. In addition, prolonged absorption of an injectable pharmaceutical form may be brought about by the inclusion of agents which delay absorption such as aluminum monostearate and gelatin. Pharmaceutical compositions are preferably manufactured under GMP conditions.

Examples of pharmaceutically-acceptable antioxidants for use in pharmaceutical compositions include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

For the therapeutic compositions, formulations for use in the methods of the present disclosure include those suitable for oral, nasal, topical (including buccal and sublingual), rectal, vaginal and/or parenteral administration. The formulations can conveniently be presented in unit dosage form and may be prepared by any methods known in the art of pharmacy. The amount of active ingredient which can be combined with a carrier material to produce a single dosage form varies depending upon the subject being treated, and the particular mode of administration. Generally, out of one hundred percent, this amount ranges from about 0.01 percent to about ninety-nine percent of active ingredient, from about 0.1 percent to about 70 percent, or from about 1 percent to about 30 percent.

Formulations of the present disclosure which are suitable for vaginal administration also include pessaries, tampons, creams, gels, pastes, foams or spray formulations containing such carriers as are known in the art to be appropriate. Dosage forms for the topical or transdermal administration of compositions of this disclosure include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

Marketed formulations of imatinib salt(s) are 100 and 400 mg film coated tablets. Many different types of Imatinib formulations have been described in the literature, including mechanically resistant tablets, different imatinib salts, enteric-coated formulations, nanoparticles, high-imatinib content tablets or different pharmaceutical forms, including solutions or aerosol formulations. None of these formulations provided for an acid resistant capsule comprising Imatinib mesylate, as instantly claimed.

Clinical studies with imatinib when administered to patients have a significant number of adverse events. The most common adverse events overall were gastrointestinal issues, such as nausea (imatinib 44.7% vs. placebo 9.2%), peripheral oedema (imatinib 26.2% vs. placebo 7.1%), vomiting (imatinib 21.4% vs. placebo 2.0%), and diarrhea (imatinib 17.5% vs. placebo 4.1%). Therefore, a need exists to formulate imatinib or a pharmaceutical salt thereof in acid resistant formulation which would delay the release of the active ingredient to avoid these adverse events.

Pharmaceutical capsules are widely used in the pharmaceutical field as oral dosage forms for administration to humans and animals. For some applications, it is desirable that the capsules be acid resistant in order to remain intact in the stomach of patients and not to release the encapsulated content therein. Acid resistant capsules are thus useful for the administration of substances that are unstable in an acidic environment, or substances that are associated with serious gastric side-effects. The acid-resistant capsules may be used in any application for solid oral dosage forms in which it is advantageous to delay release of the medicament or other material in the stomach but provide release in the intestines. One such application is the delivery of medicaments that are unstable in gastric or acidic media. Another such application is to reduce gastric side effects associated with the delivery of the medicament, such as irritation, erosion, inflammation, ulcerations, pain, reflux, and other undesirable effects. Another such application is the targeted delivery of the medicament or other material to the intestines.

Acid resistance may be achieved by employing hard pharmaceutical capsules which are generally manufactured by using a dip molding process. In this process, pin molds are dipped into an aqueous-based film-forming composition.

Also, acid resistance can be achieved with acid resistant capsules comprising hard shells wherein the acid resistance and/or enteric properties thereof are improved and/or retained (i.e. not adversely affected) even at higher pH such as when administering the capsule with water, further described in for example US 20190240160, herein incorporated by reference. These acid resistant capsule contains at least three materials: an enteric polymer having acid groups; a film-forming aid; and an alkaline material. The acid resistant capsules have improved acid resistance due to the removal or neutralization of the alkaline material in the capsule after formation of the capsule shell. The alkaline material is present in the final capsule in an amount such that the acid groups of the enteric polymer have a degree of ionization of less than 15%. The degree of ionization may preferably be less than 12%, preferably less than 10%, more preferably from 0.1% to 9%.

The acid resistance of a capsule may be achieved by coating a non-acid resistant capsule, preferably a hard shell capsule, with an enteric film. The enteric film may include acid resistant materials that have a pH-dependent water solubility. Typically, these materials are carboxylic group-containing polymers, such as cellulose acetate phthalate (CAP), hydroxypropyl methylcellulose phthalate (HPMCP), hydroxypropyl methylcellulose acetate succinate (HPMCAS), carboxyl-containing acrylic copolymers and shellac. The enteric polymer is selected from the group consisting of hydroxypropyl methylcellulose acetate succinate (HPMCAS), hydroxypropyl methylcellulose phthalate (HPMCP), cellulose acetate phthalate (CAP), acrylic polymers, polyvinyl acetate phthalate (PVAP), and mixtures thereof. Preferred enteric polymers for use herein are selected from the group consisting of HPMCAS, HPMCP, and CAP, preferably, the enteric polymer is HPMCAS. The acid groups on the enteric polymer are carboxylic acid groups. These materials are water insoluble under gastric conditions (conventionally simulated by pH 1.2) and readily water soluble under intestinal conditions (conventionally simulated by a pH of 6.8). The enteric polymer is present in a sufficient amount so as to provide the desired level of acid resistance. Typically, the enteric polymer is present in the finished capsule in an amount of from 40 to 90 wt % and preferably 50 to 80 wt %.

The acid groups on the enteric polymer in the finished acid resistant capsule may have a degree of ionization that is less than 15%. In cases where one or more enteric polymers are present, the degree of ionization value is taken as the weighted sum of the individual degree of ionization values from each polymer, where the weighting of each value is given by the fractional amount of each polymer with respect to the total amount of enteric polymer present in the composition. To determine the degree of ionization of the enteric polymer in the finished capsule, a pH is determined by dissolving finished capsule(s) to a concentration of 10 mg/mL in a stirring solution of 1:1 MeOH:H2O at 23 degrees Celsius. and then measuring the pH of the resulting solution with a pH electrode previously calibrated with pH 4.0, 7.0, and 10.0 standard buffers. It is assumed that the ratio of ionized to non-ionized acidic groups on the polymer does not change upon dissolution into a non-buffered solvent. This pH value is used in the equation above. Similarly, to determine the degree of ionization of the enteric polymer in the aqueous solution, the pH of the aqueous solution is measured, and that value is used in the equation above.

The acid-resistant capsules may be used to delay the release of one or more medicaments contained in said capsule when contacted with unbuffered water, preferably such that less than 20 wt % of said medicament is released after 60 minutes in demineralized water. Further, the acid-resistant capsules provide improved resistance to gastric dissolution media, whether in vivo or in vitro, relative to gelatin capsules and capsules formed from non-enteric polymers such as HPMC. The capsule shells comprise a dissolution release of less than about 10 wt % of the total encapsulated medicament after a time of about 2 hours when exposed to a simulated gastric media of about pH 1.2 held at a temperature of 37 degrees Celsius in which the capsule is mounted in a sinker and placed into a USP II dissolution apparatus with the paddle speed set at 50 rpm.

The acid-resistant capsules dissolve or disintegrate when exposed to intestinal buffer media, whether in vivo or in vitro, so as to rapidly release the encapsulated medicament. In one embodiment, the dissolution release is about 80 wt % of the total encapsulated medicament a time of about 45 minutes after administration to simulated intestinal buffer media of about pH 6.8 held at a temperature of 37 degrees Celsius in which the capsule is mounted in a sinker and placed into a USP II dissolution apparatus with the paddle speed set at 50 rpm.

Once filled, the capsules can be made tamper-proof by using any conventionally used technique in the field of hard capsules to make the joint permanent. Banding or sealing are suitable techniques. Sealing is a technique well known in the field of hard shell capsules. Various alternative techniques are currently used for this purpose.

The phrases “parenteral administration” and “administered parenterally” mean modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

Therapeutic compositions can be administered with medical devices known in the art. For example, in a preferred embodiment, a therapeutic composition of the disclosure can be administered with a needleless hypodermic injection device, such as the devices disclosed in, e.g., U.S. Pat. Nos. 5,399,163, 5,383,851, 5,312,335, 5,064,413, 4,941,880, 4,790,824, or 4,596,556. Examples of implants and modules useful in the present disclosure include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Many other such implants, delivery systems, and modules are known.

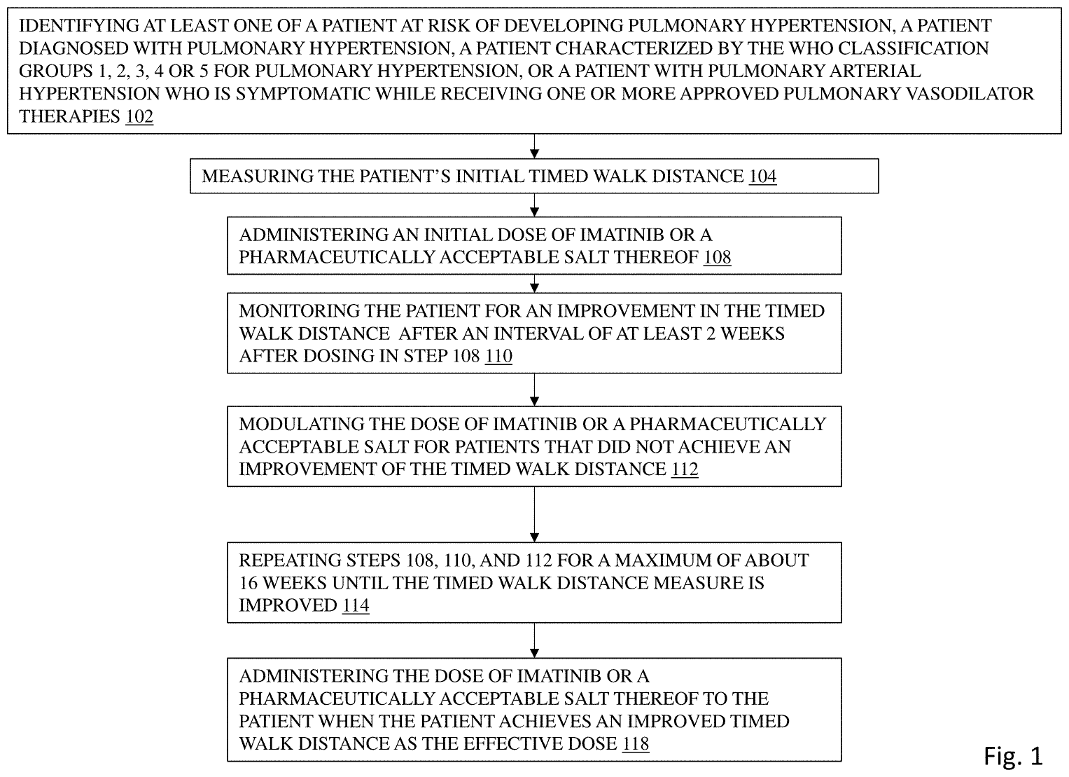

In a first aspect, and referring to FIG. 1, the present disclosure concerns a method of improving a timed walking distance in a patient suffering from pulmonary hypertension. In embodiments, the timed walking distance may be a 6-minute walking distance and the improvement may be a distance of greater than about 35 meters. It should be understood that throughout this specification, reference is made in various embodiments and examples to a 6-minute walk or walking distance as well as improvements in an amount greater than about 35 meters, however, it should be understood that in any of the embodiments and examples of this disclosure, walks of any duration and distance may be utilized, and any improvement in the distance may be used.

In embodiments, certain methods may include (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies 102; (b) measuring the patient's initial timed walk distance 104; (c) administering an initial dose of Imatinib or a pharmaceutically acceptable salt thereof 108; (d) monitoring the patient for an improvement in the timed walk distance (e.g. such as an improvement of greater than about 35 meters) after an interval of at least 2 weeks after dosing in step (c) 110; (e) modulating the dose of Imatinib or a pharmaceutically acceptable salt for patients that did not achieve an improvement of the timed walk distance of greater than about a defined distance (e.g. such as 35 meters) in step (d) 112; (f) repeating steps (d)-(e) for a maximum of about 16 weeks until the timed walk distance measure is greater than about 35 meters 114; and (g) administering the dose of Imatinib or a pharmaceutically acceptable salt thereof to the patient when the patient achieves a timed walk distance of greater than about 35 meters as the effective dose to improve a timed walk distance of greater than about 35 meters 118.

In a second aspect, the present disclosure concerns a method of improving a six minute walking distance in a patient suffering from pulmonary hypertension by greater than about 45 meters, comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) measuring the patient's initial 6-minute walk distance; (c) administering an initial dose of Imatinib or a pharmaceutically acceptable salt thereof; (d) monitoring the patient for an improvement in the 6-minute walk distance of greater than about 45 after an interval of at least 2 weeks after dosing in step (c); (e) modulating the dose of Imatinib or a pharmaceutically acceptable salt for patients that did not achieve an improvement of the 6-minute walk distance of greater than about 45 meters in step (d); (f) repeating steps (d)-(e) for a maximum of about 16 weeks until the 6-minute walk distance measure is greater than about 45 meters; and (g) administering the dose of Imatinib or a pharmaceutically acceptable salt thereof to the patient when the patient achieves a 6-minute walk distance of greater than about 45 meters as the effective dose to improve a 6-minute walk distance of greater than about 45 meters.

In a third aspect, the present disclosure concerns a method of treating pulmonary hypertension by improving a 6-minute walking distance in a patient suffering from pulmonary hypertension by greater than about 35 meters, comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) measuring the patient's initial 6-minute walk distance; (c) administering an initial dose of Imatinib or a pharmaceutically acceptable salt thereof; (d) monitoring the patient for an improvement in the 6-minute walk distance of greater than about 35 after an interval of at least 2 weeks after dosing in step (c); (e) modulating the dose of Imatinib or a pharmaceutically acceptable salt for patients that did not achieve an improvement of the 6-minute walk distance of greater than about 35 meters in step (d); (f) repeating steps (d)-(e) for a maximum of about 16 weeks until the 6-minute walk distance measure is greater than about 35 meters; and (g) administering the dose of Imatinib or a pharmaceutically acceptable salt thereof to the patient when the patient achieves a 6-minute walk distance of greater than about 35 meters as the effective dose to treat pulmonary hypertension.

In a fourth aspect, the present disclosure concerns a method of treating pulmonary hypertension by improving a six minute walking distance in a patient suffering from pulmonary hypertension by greater than about 45 meters, comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) measuring the patient's initial 6-minute walk distance; (c) administering an initial dose of Imatinib or a pharmaceutically acceptable salt thereof; (d) monitoring the patient for an improvement in the 6-minute walk distance of greater than about 45 after an interval of at least 2 weeks after dosing in step (c); (e) modulating the dose of Imatinib or a pharmaceutically acceptable salt for patients that did not achieve an improvement of the 6-minute walk distance of more than about 45 meters in step (d); (f) repeating steps (d)-(e) for a maximum of about 16 weeks until the 6-minute walk distance measure is greater than about 45 meters; and (g) administering the dose of Imatinib or a pharmaceutically acceptable salt thereof to the patient when the patient achieves a 6-minute walk distance of greater than about 45 meters as the effective dose to treat pulmonary hypertension.

In a fifth aspect, the present disclosure concerns a method of identifying patient specific biomarkers for patients with pulmonary hypertension that are responsive to Imatinib or a pharmaceutically acceptable salt thereof treatment, comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) obtaining a biological sample from said patient and determine levels of biomarkers selected from ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, FGFb, DDR1, and DDR2; (c) administering an initial dose of Imatinib or a pharmaceutically acceptable salt thereof to the patient the according to a dosing regimen; (d) monitoring the patient for a measurable response outcome after at least 2 weeks; (e) obtaining a biological sample from the patient who achieved an improved measurable response outcome and the patient who did not achieve an improved measurable response outcome and compare the biomarker levels to the sample obtained in (b); (f) identifying the modulated biomarkers in the patients who achieved an improved measurable response outcome a predictor of a responsive patient to Imatinib or a pharmaceutically acceptable salt thereof treatment for pulmonary hypertension; and optionally (g) administering to the responsive patient or predicted responsive patient having the modulated biomarkers identified in step (f) an effective dose of Imatinib of a pharmaceutically acceptable salt thereof to the patient to maintain the modulated biomarkers levels to treat pulmonary hypertension.

In a sixth aspect, the present disclosure concern a method of treating a pulmonary hypertension in a patent comprising (i) identifying patient specific biomarkers for patients with pulmonary hypertension that are responsive to Imatinib or a pharmaceutically acceptable salt thereof treatment, comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) obtaining a biological sample from said patient and determine one or more of levels of biomarkers, either protein or RNA transcript or both, selected from ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, FGFb, DDR1, and DDR2; (c) administering a dose of Imatinib or a pharmaceutically acceptable salt thereof to the patient the according to a dosing regimen; (d) monitoring the patient for a measurable response outcome after at least 2 weeks; (e) obtaining a biological sample from the patient who achieved an improved measurable response outcome and the patient who did not achieve an improved measurable response outcome and compare the biomarker levels to the sample obtained in (b); (f) identifying the modulated biomarkers in the patients who achieved an improved measurable response outcome as predictor of a responsive patient to Imatinib or a pharmaceutically acceptable salt thereof treatment for pulmonary hypertension; and (g) identifying the effective dose as the dose when the responsive patient achieves a modulated biomarkers levels; and (ii) administering to the responsive patient and/or predicted responsive patient having the modulated biomarkers identified in step (i) an effective dose of Imatinib of a pharmaceutically acceptable salt thereof to the patient to maintain the modulated biomarkers levels to treat pulmonary hypertension.

In a seventh aspect, the present disclosure concerns a method of identifying patient specific genetic signature for patients with pulmonary hypertension that are responsive to Imatinib or a pharmaceutically acceptable salt thereof treatment, comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) obtaining a biological sample from said patient and determining a genetic signature of the patient selected from one or more of ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, FGFb, DDR1, and DDR2, DSG2, EPDR1, SCD5, MGAT5, RHOQ, P2RY5, ZNF652, RALGPS2, MKNL1, RAPFEF2, TPD52, PIAS, HPRT, TPD52, ADCY8, COL15A1, TGFB1, MYH13, MTFR1, ANKRD6, DNAH1, FER1L5, ZDHHC20, CDH23, CRCP, WNT5A, and WIF1; (c) administering a dose of Imatinib or a pharmaceutically acceptable salt thereof to the patient the according to a dosing regimen; (d) monitoring the patient for a measurable response outcome after at least 2 weeks; (e) obtaining a biological sample from the patient who achieved an improved measurable response outcome and the patient who did not achieve an improved measurable response outcome and comparing the biomarker levels to the sample obtained in (b); (f) identifying the genetic signatures in the patients who achieved an improved measurable response outcome as predictor of a responsive patient to Imatinib or a pharmaceutically acceptable salt thereof treatment for pulmonary hypertension; and optionally (g) administering to the responsive patients and/or predicted responsive patient having the genetic signatures identified in step (f) an effective dose of Imatinib of a pharmaceutically acceptable salt thereof to the patient to treat pulmonary hypertension.

In an eighth aspect, the present disclosure concerns a method of treating pulmonary hypertension in a patient comprising: (i) identifying patient specific genetic signature for patients with pulmonary hypertension that are responsive to Imatinib or a pharmaceutically acceptable salt thereof treatment, comprising (a) identifying the patient at risk of developing pulmonary hypertension, diagnosed with pulmonary hypertension, patients characterized by the WHO classification groups 1, 2, 3, 4 or 5 for pulmonary hypertension, and/or patients with pulmonary arterial hypertension who are symptomatic while receiving one or more approved pulmonary vasodilator therapies; (b) obtaining a biological sample from said patient and determine genetic signature of patient selected from one or more of ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TPH-1, c-Kit, c-ABL, FGFb, DDR1, and DDR2, DSG2, EPDR1, SCD5, MGAT5, RHOQ, P2RY5, ZNF652, RALGPS2, MKNL1, RAPFEF2, TPD52, PIAS, HPRT, TPD52, ADCY8, COL15A1, TGFB1, MYH13, MTFR1, ANKRD6, DNAH1, FER1L5, ZDHHC20, CDH23, CRCP, WNT5A, and WIF1; (c) administering a dose of Imatinib or a pharmaceutically acceptable salt thereof to the patient the according to a dosing regimen; (d) monitoring the patient for a measurable response outcome after at least 2 weeks; (e) obtaining a biological sample from the patient who achieved an improved measurable response outcome and the patient who did not achieve an improved measurable response outcome and comparing the genetic signatures to the sample obtained in (b); (f) identifying the modulated genetic signatures in the patients who achieved an improved measurable response outcome as predictor of a responsive patient to Imatinib or a pharmaceutically acceptable salt thereof treatment for pulmonary hypertension; and (g) identifying the effective dose as the dose when the responsive patient achieves an improved measurable response outcome; and (ii) administering to the responsive patient and/or predicted responsive patient having the genetic signature identified in step (i) an effective dose of Imatinib of a pharmaceutically acceptable salt thereof to the patient to treat pulmonary hypertension.

In the ninth aspect, the present disclosure concerns an acid resistant capsule comprising (a) a capsule comprising at least one enteric polymer; and (b) a therapeutically effective amount of Imatinib or a pharmaceutically acceptable salt thereof. In certain embodiments of the disclosure, the enteric polymer has a degree of ionization less than about 15%, preferably be less than 12%, preferably less than 10%, more preferably from 0.1% to 9%.

In the tenth aspect, the present disclosure concerns a pharmaceutical composition comprising an acid resistant capsule comprising (a) a capsule comprising at least one enteric polymer; and (b) a therapeutically effective amount of imatinib or a pharmaceutically acceptable salt thereof; and (c) one or more pharmaceutically acceptable carrier(s). In certain embodiments of the disclosure, the enteric polymer has a degree of ionization less than about 15%, preferably be less than 12%, preferably less than 10%, more preferably from 0.1% to 9%.

In embodiments, a dosing regimen or strategy are described herein. Methods are described for treating pulmonary hypertension involving the administration of an initial dose of Imatinib or a pharmaceutically acceptable carrier to a subject suffering from pulmonary hypertension for a certain interval of time to achieve an improved measurable response outcome. If the measurable response outcome is not improved, the dose may be modulated for another interval of time until the measure response outcome is improved. Once the dosage that achieves the improved measurable response outcome is identified, this dose then becomes the effective dose to achieve the improved measurable outcome and to treat pulmonary hypertension. The dose may also be used as the maintenance dose to treat the subject. The maintenance dose may be administered daily from a minimum of about 100 mg/day to a maximum dose of 500 mg/day, preferably about 120 mg/day to about 480 mg/day. The maintenance dose may be administered at about 120 mg/day once, twice, three or four times per day.

A specific embodiment of the disclosure relates to the dosing regimen to treat pulmonary hypertension comprising (a) identifying patients at risk of developing pulmonary hypertension, diagnosed with pulmonary hypertension, patients characterized by the WHO classification groups 1, 2, 3, 4 or 5 for pulmonary hypertension, or patients with pulmonary arterial hypertension who are symptomatic while receiving one or more approved pulmonary vasodilator therapies; (b) administering a dose of Imatinib or a pharmaceutically acceptable salt thereof, which dose may be from about 100 mg/day to about 500 mg/day, preferably about 100 mg/day to about 150 mg/day, most preferably about 120 mg/day; (c) dosing for an interval of at least 2 weeks to about 5 weeks, preferably at least two weeks to at least 4 weeks, most preferably at least 4 weeks; (d) monitoring the patient for adverse events and measurable response outcomes: (e) advancing one of the following based on step (d): (i) if the patient does not tolerate the dose, the patient withdraws from the treatment; (ii) if the patient tolerates the dose and has an improved measurable response outcome to the dose, for example, the six-minute walk distance (6 MWD) test is 35 meters or greater compared to the distance measured pre-treatment, the patient continues at this dose as the chronic or maintenance dose to achieve an improvement in a measurable response outcome of the patient suffering from pulmonary hypertension. Additionally, the dose may be administered as the chronic or maintenance effective dose to treat pulmonary hypertension in the patient; and (iii) if the patient tolerates the dose, but does not improve the measurable response outcome, for examples fails to achieve a 6 minute walk which is not at least 35 meters more than the distance measured pre-treatment, then the dose administered to that patient is modulated and steps (d)-(e) are repeated at doses which may be from about 225 mg/day to about 500 mg/day, preferably 240 mg/day to about 350 mg/day, most preferably about 240 mg/day; or about 250 mg/day to about 500 mg/day, preferably 325 mg/day to about 360 mg/day, most preferably about 360 mg/day; or about 360 mg/day to about 500 mg/day, preferably 375 mg/day to about 480 mg/day, most preferably about 480 mg/day, until the patient achieves an improved measurable response outcome, for example a 6 minute walk of greater than at least 35 meters compared to the distance measured pre-treatment. The effective dose to improve the 6-minute walking distance is the dose at which the patient achieves a 6-minute walking distance of greater than about 35 meters. The effective dose may be used as a maintenance dose for the patient to maintain a 6-minute walking distance of greater than about 35 meters. The patient may not receive a dose greater than 500 mg/day, therefore, for instance where the maximum dose has been reached and the patient fails to achieve an improved measurable outcome, for example a 6-minute walk test of greater than at least 35 meters compared to pre-treatment, the treatment is withdrawn.

A specific embodiment of the dosing regimen is exemplified by the flow of the events outlined herein. About 120 mg/day imatinib for approximately 4 weeks is administered to a patient suffering from pulmonary hypertension. If the patient tolerates the dose, and if the 6 MWD increases >45 meters, the patient continues at this dose as the effective dose. If the patient tolerates the dose, and if the 6 MWD increases <45 meters, the patient is moved on to up-titration at the next dose. If the patient does not tolerate the dose, then the patient is withdrawn from treatment.

In another embodiment, about 240 mg (about 120 mg BID)/day for approximately 4 weeks is administered to a patient suffering from pulmonary hypertension. If the patient tolerates the dose, and if the 6 MWD increases >45 meters, the patient continues at this dose as the effective dose. If the patient tolerates the dose, and if the 6 MWD increases <45 meters, the patient is moved on to up-titration at the next dose. If the patient does not tolerate the dose, but clinically improves (increased 6 MWD>35 meters), the patient is down-titrated to a lower dose for 4 more weeks. If the patient does not tolerate the dose, and is not clinically improved, then the patient is withdrawn from treatment.

In another embodiment, about 360 mg (about 120 mg TID)/day for approximately 4 weeks is administered to a patient suffering from pulmonary hypertension. If the patient tolerates the dose, and if the 6 MWD increases >45 meters, the patient continues at this dose as the effective dose. If the patient tolerates the dose, and if the 6 MWD increases <45 meters, the patient is moved on to up-titration at the next dose. If the patient does not tolerate the dose, but clinically improves (increased 6 MWD>35 meters), the patient is down-titrated to lower dose for 4 more weeks. If the patient does not tolerate the dose, and is not clinically improved, then the patient is withdrawn from treatment.

In another embodiment, about 480 mg (divided doses of 120 mg four times per day)/day for approximately 4 weeks is administered to a patient suffering from pulmonary hypertension. If the patient tolerates the dose, and if the 6 MWD increases >45 meters, the patient continues at this dose as the effective dose. If the patient tolerates the dose, but 6 MWD increases <45 meters, then the patient is withdrawn from treatment. If the patient does not tolerate the dose, then the patient is withdrawn from treatment.

In certain embodiments of the disclosure, the dose range of Imatinib or a pharmaceutically acceptable salt thereof to treat pulmonary hypertension, including all sub-types, and to improve measurable response outcomes in patients suffering from pulmonary hypertension includes from about 100 mg/day to about 500 mg/day, preferably, from about 120 mg/day to about 480 mg/day, most preferably about 120 mg/day, about 240 mg/day, about 360 mg/day, or about 480 mg/day. The dose may also be administered at about 120 mg at an interval of once, twice, three or four times per day. The doses may also include 120 mg/day, 125 mg/day, 130 mg/day, 140 mg/day, 150 mg/day, 160 mg/day, 170 mg/day, 180 mg/day, 190 mg/day, 200 mg/day, 210 mg/day, 220 mg/day, 230 mg/day, 240 mg/day, 250 mg/day, 260 mg/day, 270 mg/day, 280 mg/day, 290 mg/day, 300 mg/day, 310 mg/day, 320 mg/day, 330 mg/day, 340 mg/day, 350 mg/day, 360 mg/day, 370 mg/day, 380 mg/day, 390 mg/day, 400 mg/day, 410 mg/day, 420 mg/day, 430 mg/day, 440 mg/day, 450 mg/day, 460 mg/day, 470 mg/day, and 480 mg/day.

Depending on species, age, individual condition, mode of administration, and the clinical picture in question, effective doses may be, for example, daily doses of about 100 mg/day to about 500 mg/day, preferably, from about 120 mg/day to about 480 mg/day, most preferably about 120 mg/day, about 240 mg/day, about 360 mg/day, or about 480 mg/day. The dose of Imatinib or a pharmaceutically acceptable salt may also be administered at about 120 mg at an interval of once, twice, three or four times per day to warm-blooded animals of about 70 kg bodyweight. For adult patients, a starting dose corresponding to about 120 mg/day of Imatinib free base or a pharmaceutically acceptable salt thereof daily may be recommended. For patients with an inadequate measurable response outcome after an assessment of response to therapy with a dose corresponding to about 120 mg/day of imatinib free base or a pharmaceutically acceptable salt thereof daily, dose escalation regimen can be administered until adequate measurable response outcome is achieved and patients may be treated as long as they benefit from treatment and in the absence of limiting toxicities.

In certain embodiments of the present disclosure, the dosage interval for the dosing regimen includes and may be between about 1 week to no more than 20 weeks, preferably about 2 weeks to about 16 weeks, most preferably about 4 weeks to about 16 weeks. Each successive modulated dose may be administered over intervals of about 1 week to no more than about 5 weeks, preferably of about 2 weeks to about 4 weeks, and most preferably of about 4 weeks. The interval may include about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 16 weeks. The interval may also include 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, and 16 weeks.

In certain embodiments of the present disclosure, the measurable outcomes for the dosing regimen may include primarily an improved in 6-minute walk distance measure, and secondarily by improvement in clinical status (assessment of WHO class and Borg Score), and changes in pulmonary hemodynamic parameters (including mean pulmonary arterial pressure, mean Pulmonary Artery Wedge pressure, Systolic Arterial Pressure, Heart Rate, and Cardiac Output, Pulmonary Vascular Resistance, Systemic Vascular Resistance), time to clinical worsening, changes in plasma biomarker levels, and any physical test. The physical test components may include any type of measurable physical activity, measurable by time, distance or physical parameters, such as heart rate, blood pressure, etc.

In certain embodiments of the present disclosure, the primary measurable response outcome includes an about 6-minute walk measured by the distance walked in meters. The measurable response outcomes of a 6-minute walk test includes a measurable distance of greater than about 6 meters, greater than about 10 meters, greater than about 15 meters, greater than about 20 meters, greater than about 25 meters, greater than about 30 meters, greater than about 35 meters, greater than about 40 meters and greater than about 45 meters compared to the six minute walk distance measured distance pre-treatment. It could also include a 6-minute walk distance measured as greater than about 6 meters, about 7 meters, about 8 meters, about 9 meters, about 10 meters, about 11 meters, about 12 meters, about 13 meters, about 14 meters, about 15 meters, about 16 meters, about 17 meters, about 18 meters, about 19 meters, about 20 meters, about 21 meters, about 22 meters, about 23 meters, about 24 meters, about 25 meters, about 26 meters, about 27 meters, about 28 meters, about 29 meters, about 30 meters, about 31 meters, about 32 meters, about 33 meters, about 34 meters, about 35 meters, about 36 meters, about 37 meters, about 38 meters, about 39 meters, about 40 meters, about 41 meters, about 42 meters, about 43 meters, about 44 meters, or about 45 meters compared to the 6-minute walk distance measured distance pre-treatment.

In certain embodiments of the present disclosure, an improvement to the measurable response outcome includes the distance achieved in the at least 6-minute walk is at least about 35 meters or greater than the measured distance pre-treatment in patient suffering from pulmonary hypertension. An improved measurable response outcome and/or a measurable response outcome that is improved includes an indication of an effective dose to treat the disease, pulmonary hypertension.

In certain embodiments of the present disclosure, an improved measurable response outcome includes when the patient achieves at least about 35 meters or greater than the distance in an at least 6-minute walk compared to the distance measured prior to treatment. The improved measurable response outcome distance improvement may be at least about 45 meters or more, preferably, at least about 40 meters or more, preferably at least about 35 meters or more, at least about 30 meters or more, at least about 31 meters or more, at least about 32 meters or more, at least about 33 meters or more, at least about 34 meters or more, at least about 36 meters or more, at least about 37 meters or more, at least about 38 meters or more, at least about 39 meters or more, at least about 40 meters or more, at least about 41 meters or more, at least about 42 meters or more, at least about 43 meters or more, at least about 44 meters or more, at least about 46 meters or more, at least about 47 meters or more, at least about 48 meters or more, at least about 49 meters or more, at least about 50 meters or more.

In certain embodiments of the present disclosure, an exceptional measurable response includes when the patient achieves at least about 45 meters or greater than the distance measured in an about 6-minute walk prior to treatment.

In certain embodiments of the present disclosure, the dosing regimen includes that the patient tolerate the treatment dose being administered to treat pulmonary hypertension. In order to tolerate the treatment dose, the patient should not exhibit any serious adverse event. The “severe adverse event” (SAE) is a grade III or IV adverse event as defined by the National Cancer Institute (NCI). A grade III event is defined as severe and a grade IV event is generally defined as life-threatening or disabling.

In embodiment, proteomic and transcriptomic biomarkers may be determined. In certain embodiments of the present disclosure are methods of treating patients suffering from pulmonary hypertension, and in particular pulmonary arterial hypertension, which can be successfully treated with Imatinib or a pharmaceutically acceptable salt thereof by administering to a patient in need of such therapy the effective dose of Imatinib or a pharmaceutically acceptable salt thereof, and measuring the effectiveness with proteomic biomarkers levels and RNA signatures before modulating the dose so that the effective dose is used chronically.

In certain embodiments of the present disclosure are methods of improving the efficacy and tolerance of Imatinib or a pharmaceutically acceptable salt thereof in pulmonary hypertension, including pulmonary arterial hypertension and other forms of pulmonary hypertension, by adjusting the dose of said drug by identifying a proteomic or transcriptomic biomarker(s) that will change in parallel with the efficacy of the drug so that the dose adjustments, both higher and lower, can be made to improve efficacy and tolerance and treat pulmonary hypertension. Alternatively, there may be opposite effects such that the biomarker changes in opposite direction of the clinical efficacy of the drug.

In certain embodiments are methods to identify a unique RNA expression pattern and proteomic biomarker signature of patients with pulmonary hypertension who are Imatinib-, or a pharmaceutically acceptable salt thereof. responders by comparing the expression in responders to Imatinib or a pharmaceutically acceptable salt thereof against the expression of Imatinib or a pharmaceutically acceptable salt thereof non-responders after treatment for 16 weeks as follows. The method includes identifying the proteomic biomarkers in patients suffering from pulmonary hypertension administered Imatinib or a pharmaceutically acceptable salt thereof, such as but not limited to ABL1, ABL2, ALK1, ERBB2, ERBB3, ERBB4, EphA1, EphA3, FGFR2, FCGR3, Met, Ror1, VEGF, VEFGD, Vegfsr2, Vegsfr3, Ret, PDGF, PDGR-RB, PDGF-CC, PDGF-BB, PDGF-AA. PDF, PDFGB, bFGF, EGF, 5-HT, TSP-1, c-Kit, c-ABL, TGFb, DDR1, or DDR2. The top 25 most differentially expressed proteins from responders and non-responders are evaluated using an RNA Expression Pattern Method or a Proteomic Expression Pattern.

In the RNA Expression Pattern Method, peripheral blood lymphocytes are EBV transformed and cultured ex vivo for 6 weeks, when RNA is isolated and samples sent for RNASeq using a commercial platform. After obtaining expression data, the 25 most differentially expressed genes are identified and confirmed using qPCR. Because circulating RNA in peripheral blood is more practical for clinical use, qPCR using whole blood RNA will be used for gene expression of the genes validated using qPCR. Whole blood RNA is isolated from PAXGene tubes, in which fresh blood can be collected and stored and RNA isolated later from whole blood.

In the Proteomic Expression Pattern, the EDTA plasma collected in the general protocol is used and non-targeted proteomics using Olink panels (Olink, Watertown, Mass.)) is performed for aptamer-based identification and quantification of >1300 proteins. The top 25 most differentially expressed proteins are validated using ELISAs in remaining plasma from responders and non-responders.

In embodiments, genetic biomarkers may be identified. Identifying a genetic biomarker that is unique to patients with pulmonary hypertension who are Imatinib responders by comparing gene variant patterns of responders to Imatinib against the gene variant patterns of Imatinib non-responders after treatment for 16 weeks may proceed as follows: DNA is subjected to whole genome sequencing (WGS). After obtaining the WGS data, a genome wide association study is completed to identify SNVs associated with good clinical response to Imatinib therapy. Although the GWAS analysis may be underpowered at a stringent significance level such as 5×10⁻⁸, this exploratory analysis will allow identity of candidate SNVs associated with clinical Imatinib response. Candidate genes can also be refined by searching the Geneotype-Tissue Expression Project (GTEx) database30 using SNVs associated with Imatinib response for relevant tissue expression. Candidate genes can be further explored using pathway and gene ontology analysis. Identified SNVs can be validated by Sanger sequencing in the primary samples. SNVs can be prioritized based on the frequency of variants in the general population and the predicted effect of SNV on protein function.

In embodiments, methods may be used for identifying patients with pulmonary hypertension who would respond to treatment with Imatinib or a pharmaceutically acceptable salt thereof. Once the proteomic biomarkers and genetic signatures are identified by the methods described above, certain embodiments of the present disclosure provide methods of treating a patient with pulmonary hypertension, the method comprising the steps of: determining the genetic signature of patients with pulmonary arterial hypertension who respond effectively to Imatinib or a pharmaceutically acceptable salt thereof; determining if patients with pulmonary hypertension (other than pulmonary arterial hypertension) have a similar genetic signature common to patients with pulmonary arterial hypertension that respond effectively to Imatinib or a pharmaceutically acceptable salt thereof; and administering the effective dose of Imatinib or a pharmaceutically acceptable salt thereof to said patients with pulmonary hypertension, wherein the effective dose is determined by a dosing regimen as used in patients with pulmonary arterial hypertension.

In embodiments, methods may be used for treating patients with pulmonary hypertension chronically with adjusted lowest effective dose of Imatinib or a pharmaceutically acceptable salt thereof. For patients already on a treatment regimen for pulmonary hypertension with the effective dose previously determined by a dosing regimen, certain embodiments of the present disclosure provides for a method adjusting the effective dose for chronic management of pulmonary hypertension in a patient in need thereof, said method comprising: (a) establishing an adjusted effective dose comprising: (a) monitoring the proteomic biomarker and RNA expression biomarker signature of patients with pulmonary hypertension who are on Imatinib or a pharmaceutically acceptable salt thereof therapy to periodically detect changes in the levels of the proteomic biomarkers over time and comparing them to previous measures of clinical efficacy selected from a 6 minute walk distance measure, echocardiogram, Functional Class, Quality of Life questionnaire, and hemodynamics; (b) adjusting the Imatinib or a pharmaceutically acceptable salt thereof dose downward in patients who maintain an exceptional response when the biomarker levels fall significantly compared to previous levels shown by the same patient, indicating effective Imatinib mechanism of action, to achieve the adjusted effective dose; (c) adjusting the Imatinib or a pharmaceutically acceptable salt thereof dose upward in patients who lose an exceptional response when the biomarker levels, indicating loss of effective Imatinib mechanism of action, to improve the efficacy of treatment; and (e) administering the adjusted effective dose to chronically treat pulmonary hypertension.

In certain embodiments, the adjusted effective dose includes from about 100 mg/day to about 500 mg/day, preferably, from about 120 mg/day to about 480 mg/day, most preferably about 120 mg/day, about 240 mg/day, about 360 mg/day, or about 480 mg/day. The dose may also administer as about 120 mg administered once, twice, three or four times per day. The does may also include 120 mg/day, 125 mg/day, 130 mg/day, 140 mg/day, 150 mg/day, 160 mg/day, 170 mg/day, 180 mg/day, 190 mg/day, 200 mg/day, 210 mg/day, 220 mg/day, 230 mg/day, 240 mg/day, 250 mg/day, 260 mg/day, 270 mg/day, 280 mg/day, 290 mg/day, 300 mg/day, 310 mg/day, 320 mg/day, 330 mg/day, 340 mg/day, 350 mg/day, 360 mg/day, 370 mg/day, 380 mg/day, 390 mg/day, 400 mg/day, 410 mg/day, 420 mg/day, 430 mg/day, 440 mg/day, 450 mg/day, 460 mg/day, 470 mg/day, and 480 mg/day.

In an embodiment, a method of treating a human suffering from pulmonary arterial hypertension (PAH) may include administering a pharmaceutical composition of 4-(4-methylpiperazin-1-ylmethyl)-N-[4-methyl-3-(4-pyridin-3-yl)pyrimidin-2-ylamino)phenyl]-benzamide (imatinib), or a pharmaceutically acceptable salt thereof to a patient. The pharmaceutical composition may be formulated for delayed release in a capsule comprising at least one enteric polymer, a film-forming aid and an alkaline material. The pharmaceutical composition may be formulated to deliver a dose of at least 100 mg/day of imatinib. The at least one enteric polymer includes at least one acid group, such as a carboxylic acid group or other groups described herein. The alkaline material may be present in an amount such that a carboxylic acid group of the at least one enteric polymer has a degree of ionization of less than 15%.

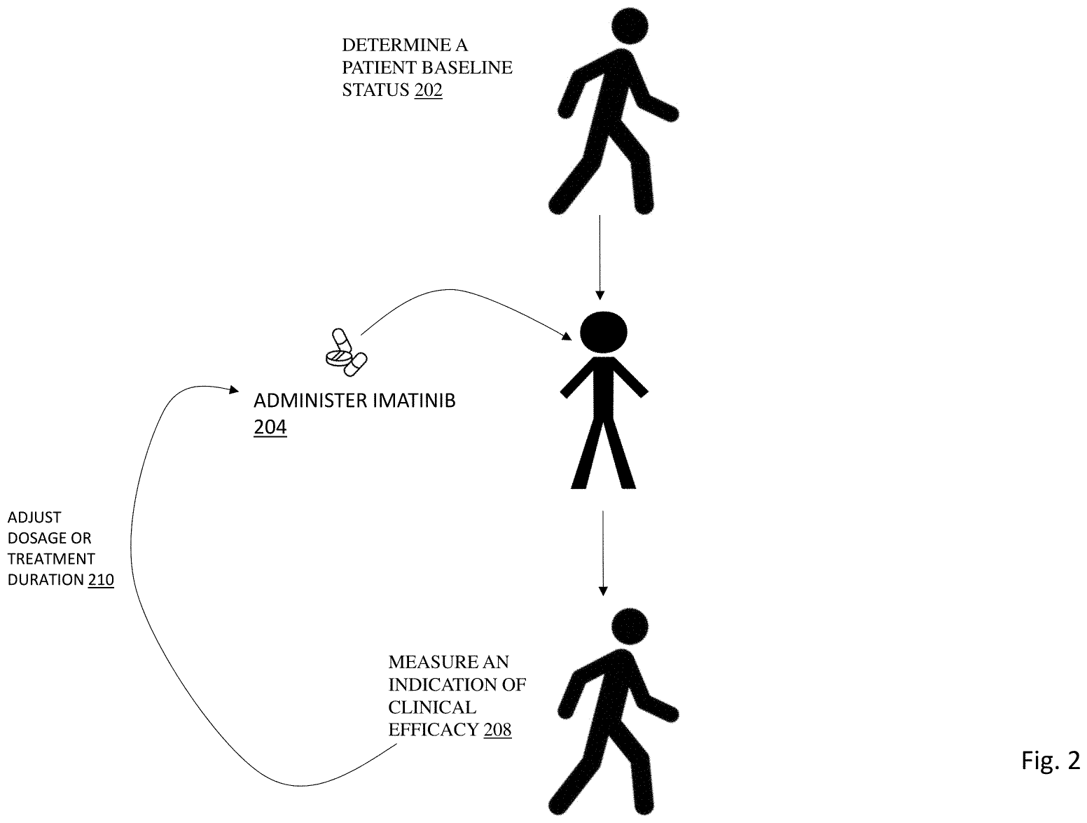

In an embodiment, a method of treating a human suffering from pulmonary arterial hypertension (PAH) may include administering a pharmaceutical composition of imatinib, or a pharmaceutically acceptable salt thereof, in an amount effective to improve an indication of clinical efficacy, wherein the pharmaceutical composition is formulated for delayed release in a capsule comprising at least one enteric polymer and an alkaline material, and wherein the pharmaceutical composition is formulated to deliver a dose of at least 100 mg/day of imatinib. Referring to FIG. 2, a patient baseline status may first be measured 202. Then, the imatinib-based pharmaceutical composition may be administered for a treatment duration 204. Clinical efficacy may be measured 208. The indication of clinical efficacy may be a change in at least one of a proteomic biomarker, an improvement in a 6-minute walking distance by greater than about 35 meters, a protein biomarker, a transcriptomic biomarker, a patient specific genetic signature, or an RNA expression biomarker signature. Based on the measurement, the dosage or treatment duration may be adjusted 210.

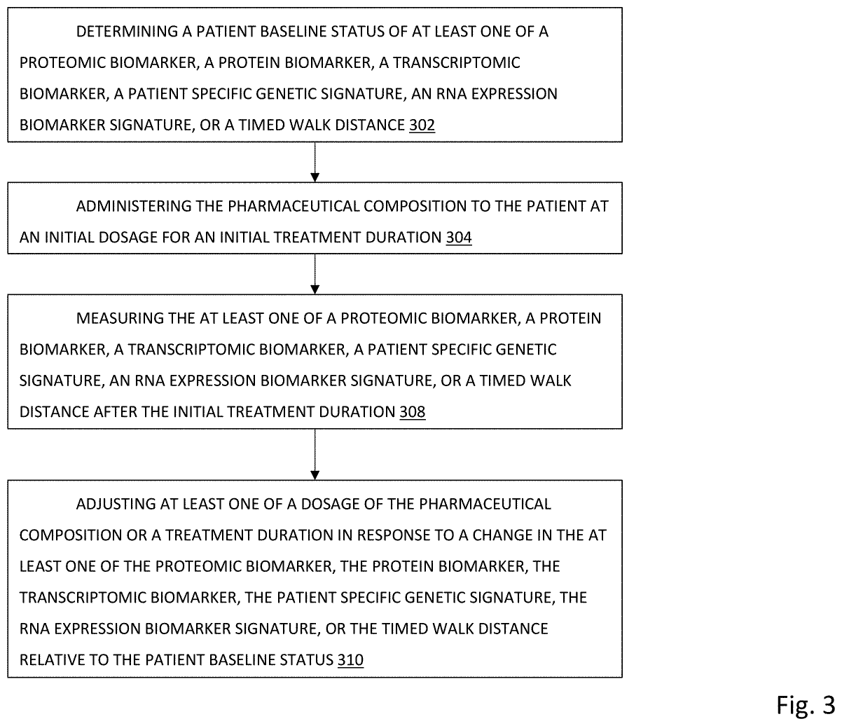

In an embodiment, and referring to FIG. 3, a method for treating a patient with a pharmaceutical composition comprising imatinib or a pharmaceutically acceptable salt thereof, wherein the patient is suffering from pulmonary arterial hypertension includes the steps of: determining a patient baseline status of at least one of a proteomic biomarker, a protein biomarker, a transcriptomic biomarker, a patient specific genetic signature, an RNA expression biomarker signature, or a timed walk distance 302 then administering the pharmaceutical composition to the patient at an initial dosage for an initial treatment duration 304. After the initial treatment duration, the at least one of a proteomic biomarker, a protein biomarker, a transcriptomic biomarker, a patient specific genetic signature, an RNA expression biomarker signature, or a timed walk distance are measured 308. The method further includes adjusting at least one of a dosage of the pharmaceutical composition or a treatment duration in response to a change in the at least one of a proteomic biomarker, a protein biomarker, a transcriptomic biomarker, a patient specific genetic signature, an RNA expression biomarker signature, or a timed walk distance relative to the patient baseline status 310. The pharmaceutical composition may be formulated for delayed release in a capsule. The capsule may include at least one enteric polymer having acid groups, a film-forming aid, and an alkaline material, wherein the alkaline material is present in an amount such that one or more carboxylic acid groups of the at least one enteric polymer has a degree of ionization of less than 15%. The alkaline material may be at least one of ammonia, ethanolamine, diethanolamine, triethanolamine, lithium hydroxide, sodium hydroxide, potassium hydroxide, calcium hydroxide, magnesium hydroxide, sodium phosphate, sodium carbonate, sodium citrate, sodium ascorbate, lysine, arginine, or cationic polymers. The at least one enteric polymer may be at least one of hydroxypropyl methylcellulose acetate succinate (HPMCAS), hydroxypropyl methylcellulose phthalate (HPMCP), cellulose acetate phthalate (CAP), acrylic polymers, or polyvinyl acetate phthalate (PVAP). The film-forming aid may be at least one of hydroxypropyl methylcellulose (HPMC), methylcellulose (MC), gellan gum, or carrageenan. The initial dosage may be at least 100 mg/day. The initial treatment duration may be at least two weeks. The pharmaceutical composition may further include at least one of a calcium channel antagonist, a prostacyclin, a prostacyclin analogues, a nonprostanoid prostacyclin receptor agonist, adenosine, inhaled nitric oxide, warfarin, digoxin, an endothelin receptor blocker, a phosphodiesterease inhibitor, norepinephrine, an angiotensin-converting enzyme inhibitor, a diuretic, ambrisentan, tadalafil, bosentan, treprostinil, macitentan, epoprostenol, iloprost, riociguat, or selexipag. In embodiments, the timed walk distance may be a 6-minute walk distance.

Adjusting the dosage of the pharmaceutical composition comprises adjusting the dosage downward if the patient exhibits an improvement as measured by the indication of clinical efficacy. The dosage continues to be adjusted downward to an effective dose where the first patient still exhibits an improvement as measured by the indication of clinical efficacy. Adjusting the dosage of the pharmaceutical composition comprises adjusting the dosage upward if the patient does not exhibit an improvement as measured by the indication of clinical efficacy. The dosage continues to be adjusted upward incrementally to an effective dose where the first patient exhibits an improvement as measured by the indication of clinical efficacy. In any event, the dosage may be adjusted to reach or maintain biomarker levels associated with effective treatment of pulmonary hypertension with imatinib.

Data regarding the indication of clinical efficacy derived during treatment for a first patient may be used to predict a response for a second patient and to set an initial, effective, or maintenance dose for a second patient. For example, patients who respond well to imatinib treatment for pulmonary hypertension may exhibit certain biomarkers (e.g. proteomic biomarker, a protein biomarker, a transcriptomic biomarker, a patient specific genetic signature, an RNA expression biomarker signature) or certain levels of biomarkers. If a second patient's biomarker baseline status is similar to a first patient who responded well to imatinib, the second patient's initial dose may be set to match the first patient's current dose or initial dose. If a second patient's biomarker baseline status is similar to a first patient who did not respond well to a dosage of imatinib, the second patient's initial dose may be increased relative to a patient with biomarker levels indicative of a good response.

In another example, the initial dosage for a patient may be determined by comparing the patient baseline status with at least one biomarker level of at least one responsive patient. Then, the initial dosage is set to be the at least one responsive patient's effective dose.

Biomarker (e.g. proteomic biomarker, a protein biomarker, a transcriptomic biomarker, a patient specific genetic signature, an RNA expression biomarker signature) levels may be monitored during treatment and may be used to adjust the dosage to maintain efficacy. Adjusting the dosage of the pharmaceutical composition comprises adjusting the dosage downward if the first patient maintains an exceptional response in the timed walk distance even when at least one of the biomarker levels falls significantly compared to a previous level shown by the first patient. Adjusting the dosage of the pharmaceutical composition comprises adjusting the dosage upward if the first patient no longer exhibits an exceptional response in the timed walk distance even when at least one of the biomarker levels is maintained or increased relative to a previous level shown by the first patient.

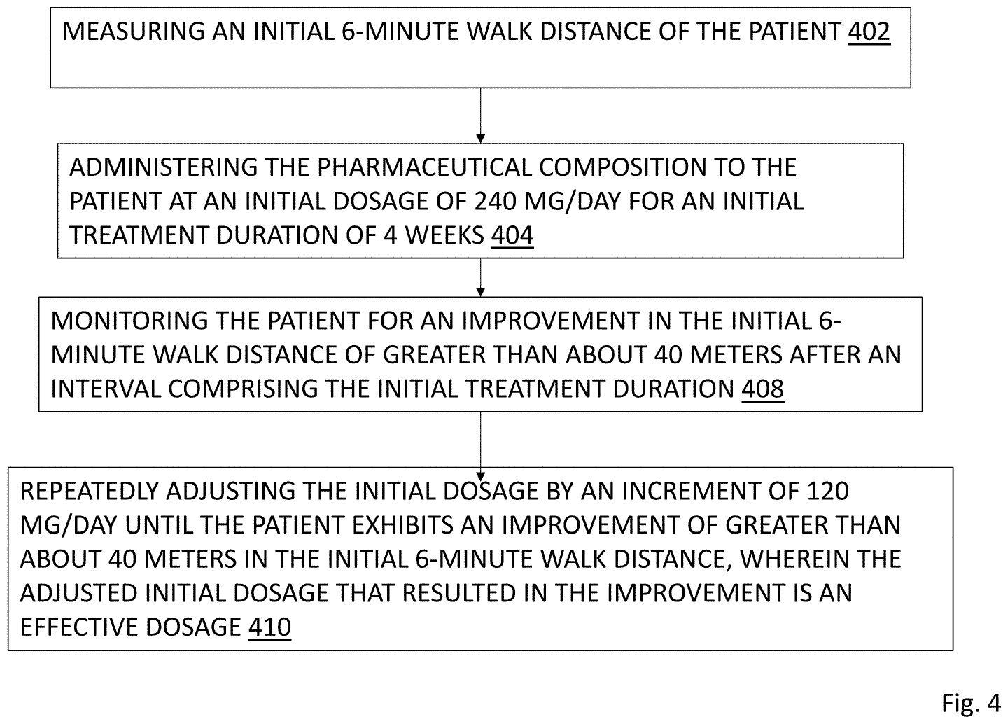

Referring now to FIG. 4, a method for treating a patient with a pharmaceutical composition comprising imatinib or a pharmaceutically acceptable salt thereof, wherein the patient is suffering from or suspected of suffering from pulmonary arterial hypertension, includes measuring an initial 6-minute walk distance of the patient 402, administering the pharmaceutical composition to the patient at an initial dosage of 240 mg/day for an initial treatment duration of 4 weeks 404, monitoring the patient for an improvement in the initial 6-minute walk distance of greater than about 40 meters after an interval comprising the initial treatment duration 408, and repeatedly adjusting the initial dosage by an increment of 120 mg/day until the patient exhibits an improvement of greater than about 40 meters in the initial 6-minute walk distance, wherein the adjusted initial dosage that resulted in the improvement is an effective dosage 410. A maximum dosage of the pharmaceutical composition after repeatedly adjusting may be 480 mg/day. A maximum duration of interval of repeated adjustment without an improvement may be about 16 weeks. Patients are classified as a nonresponder if the patient does not exhibit an improvement after repeatedly adjusting the initial dosage up to a maximum dosage and as a responder if the patient exhibits an improvement.

For example, imatinib significantly improved 6 MWD at week 24 compared with patients receiving a placebo, with a mean between-group difference of 32 m. In this scenario, the dosage may be escalated by 120 mg/day to a daily dosage of 360 mg/day for an additional treatment duration (e.g. 4 weeks). After the 4 week interval of treatment, the 6 MWD may once again be measured and the improvement relative to the initial 6 MWD may be recorded. At this point, the current dose may be deemed to be an effective dose for the patient.

The circulating biomarker profile of responsive patients may be monitored. The circulating biomarker profile comprises a level or an expression pattern of at least one of a proteomic biomarker, a protein biomarker, a transcriptomic biomarker, a patient specific genetic signature, or an RNA expression biomarker signature, the circulating biomarker profile correlating with a responsiveness of the responder. The effective dosage may be adjusted upward if the circulating biomarker profile indicates a decreased responsiveness of the responder or downward if the circulating biomarker profile indicates an increased responsiveness of the responder.

A circulating biomarker profile of a second patient may be determined and compared to the circulating biomarker profile of the first patient after the treatment interval and after an effective dose has been determined. If there is at least one similarity between the circulating biomarker profile of the second patient and the circulating biomarker profile of the first patient, then the initial dosage for the second patient is set to be the effective dose of the first patient.

In an embodiment, a method for treating a patient suffering from or suspected of suffering from PAH with an imatinib pharmaceutical composition includes determining a circulating biomarker profile of the patient, the circulating biomarker profile comprising a level or an expression pattern of at least one of a proteomic biomarker, a protein biomarker, a transcriptomic biomarker, a patient specific genetic signature, or an RNA expression biomarker signature prior to administration of the pharmaceutical composition; administering the pharmaceutical composition to the first patient at an initial dosage of 240 mg for an initial treatment duration of 4 weeks and repeatedly adjusting the initial dosage by an increment of 120 mg/day for a treatment duration of 4 weeks until the first patient exhibits a change in the circulating biomarker profile indicative of a responsiveness, wherein the adjusted initial dosage that resulted in the change is an effective dosage; classifying the patient as a responder if the patient exhibits a change; determining the circulating biomarker profile after a treatment interval with the effective dosage; and adjusting the effective dosage if the circulating biomarker profile begins to revert to a pre-administration level or expression pattern.

In an embodiment, a method for treating a patient suffering from or suspected of suffering from PAH with an imatinib pharmaceutical composition includes determining a circulating biomarker profile of the patient, the circulating biomarker profile comprising a level or an expression pattern of at least one of a proteomic biomarker, a protein biomarker, a transcriptomic biomarker, a patient specific genetic signature, or an RNA expression biomarker signature prior to administration of the pharmaceutical composition; comparing the circulating biomarker profile to a plurality of responsive patient circulating biomarker profiles and, if there is at least one match, administering the pharmaceutical composition to the first patient at a dosage for a treatment interval; and monitoring the circulating biomarker profile after the treatment interval, and adjusting the dosage if the circulating biomarker profile no longer matches at least one of the plurality of responsive patient circulating biomarker profiles. The dosage may be set to be an effective dosage for at least one responsive patient whose responsive patient circulating biomarker profile matched the circulating biomarker profile. Alternatively, the dosage may be 240 mg/day for an initial treatment duration of 4 weeks. The dosage may be repeatedly adjusted by an increment of 120 mg/day for a treatment duration of 4 weeks until the patient exhibits an improvement of greater than about 40 meters in a 6-minute walk distance.

In an embodiment, a method for treating a patient suffering from or suspected of suffering from PAH with an imatinib pharmaceutical composition includes determining a patient specific genetic signature prior to administration of the pharmaceutical composition; comparing the circulating biomarker profile to a plurality of responsive patient circulating biomarker profiles and, if there is at least one match, administering the pharmaceutical composition to the first patient at a dosage for a treatment interval; and monitoring the circulating biomarker profile after the treatment interval, and adjusting the dosage if the circulating biomarker profile no longer matches at least one of the plurality of responsive patient circulating biomarker profiles. The dosage may be set to be an effective dosage for at least one responsive patient whose responsive patient circulating biomarker profile matched the circulating biomarker profile. Alternatively, the dosage may be 240 mg/day for an initial treatment duration of 4 weeks. The dosage may be repeatedly adjusted by an increment of 120 mg/day for a treatment duration of 4 weeks until the patient exhibits an improvement of greater than about 40 meters in a 6-minute walk distance.

As with any embodiment disclosed herein, the pharmaceutical composition may be formulated for delayed release in a capsule, wherein the capsule comprises at least one enteric polymer having acid groups, a film-forming aid, and an alkaline material. The alkaline material may be present in an amount such that one or more carboxylic acid groups of the at least one enteric polymer has a degree of ionization of less than 15%. The alkaline material may be at least one of ammonia, ethanolamine, diethanolamine, triethanolamine, lithium hydroxide, sodium hydroxide, potassium hydroxide, calcium hydroxide, magnesium hydroxide, sodium phosphate, sodium carbonate, sodium citrate, sodium ascorbate, lysine, arginine, or cationic polymers. The at least one enteric polymer may be at least one of hydroxypropyl methylcellulose acetate succinate (HPMCAS), hydroxypropyl methylcellulose phthalate (HPMCP), cellulose acetate phthalate (CAP), an acrylic polymer, or polyvinyl acetate phthalate (PVAP). The film-forming aid may be at least one of hydroxypropyl methylcellulose (HPMC), methylcellulose (MC), gellan gum, or carrageenan. In an example, the capsule may include 65% HPMCAS, 28% HPMC, 0.2% ammonia, 2% Titanium dioxide, and 4.8% water, and is filled with 120 mg imatinib or a pharmaceutically acceptable salt thereof (e.g. imatinib mesylate). In a further example, the capsule may include 64% HPMCP, 29% MC, 0.3% ammonia, 2% Titanium dioxide, and 4.7% water, and is filled with 120 mg imatinib or a pharmaceutically acceptable salt thereof (e.g. imatinib mesylate). In any embodiment, the imatinib or a pharmaceutically acceptable salt thereof may be formulated with excipients or other inert fillers.

The pharmaceutical composition may further include at least one of a calcium channel antagonist, a prostacyclin, a prostacyclin analogues, a nonprostanoid prostacyclin receptor agonist, adenosine, inhaled nitric oxide, warfarin, digoxin, an endothelin receptor blocker, a phosphodiesterease inhibitor, norepinephrine, an angiotensin-converting enzyme inhibitor, a diuretic, ambrisentan, tadalafil, bosentan, treprostinil, macitentan, epoprostenol, iloprost, riociguat, or selexipag.

The present disclosure is not limited to any particular preferred embodiment(s) described herein. Indeed, many modifications and variations of the disclosure will be apparent to those skilled in the art upon reading this specification, and such “equivalents” can be made without departing from the disclosure in spirit or scope. The disclosure is therefore limited only by the terms of the appended claims, along with the full scope of equivalents to which the claims are entitled.

While only a few embodiments of the present disclosure have been shown and described, it will be obvious to those skilled in the art that many changes and modifications may be made thereunto without departing from the spirit and scope of the present disclosure as described in the following claims. All patent applications and patents, both foreign and domestic, and all other publications referenced herein are incorporated herein in their entireties to the full extent permitted by law.

While the disclosure has been disclosed in connection with the preferred embodiments shown and described in detail, various modifications and improvements thereon will become readily apparent to those skilled in the art. Accordingly, the spirit and scope of the present disclosure is not to be limited by the foregoing examples, but is to be understood in the broadest sense allowable by law.

The use of the terms “a” and “an” and “the” and similar referents in the context of describing the disclosure (especially in the context of the following claims) is to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the disclosure, and does not pose a limitation on the scope of the disclosure unless otherwise claimed. No language in the specification should be construed as indicating any non-claimed element as essential to the practice of the disclosure.

While the foregoing written description enables one skilled in the art to make and use what is considered presently to be the best mode thereof, those skilled in the art will understand and appreciate the existence of variations, combinations, and equivalents of the specific embodiment, method, and examples herein. The disclosure should therefore not be limited by the above described embodiment, method, and examples, but by all embodiments and methods within the scope and spirit of the disclosure.

Any element in a claim that does not explicitly state “means for” performing a specified function, or “step for” performing a specified function, is not to be interpreted as a “means” or “step” clause as specified in 35 U.S.C. § 112(f). In particular, any use of “step of” in the claims is not intended to invoke the provision of 35 U.S.C. § 112(f).

Persons skilled in the art may appreciate that numerous design configurations may be possible to enjoy the functional benefits of the inventive systems. Thus, given the wide variety of configurations and arrangements of embodiments of the present disclosure, the scope of the disclosure is reflected by the breadth of the claims below rather than narrowed by the embodiments described above.

1.-99. (canceled)

100. A method for treating a first patient with a pharmaceutical composition comprising imatinib or a pharmaceutically acceptable salt thereof, wherein the first patient is suffering from pulmonary arterial hypertension, the method comprising the steps of:
determining a first status of an indication of clinical efficacy for the first patient, wherein the indication of clinical efficacy comprises a measurement related to at least one of a biomarker or a timed walk distance, wherein the biomarker is at least one of a proteomic biomarker, a protein biomarker, a transcriptomic biomarker, a patient specific genetic signature, or an RNA expression biomarker signature;
administering the pharmaceutical composition to the first patient at an initial dosage for an initial treatment duration;
measuring the at least one biomarker or timed walk distance after the initial treatment duration to obtain a second status of an indication of clinical efficacy; and
adjusting the initial dosage to obtain an adjusted dosage for the first patient if the second status of the indication of clinical efficacy is changed from the first status of the indication of clinical efficacy.

101. The method of claim 100, wherein the pharmaceutical composition is formulated for delayed release in a capsule.

102. The method of claim 101, wherein the capsule comprises:
at least one enteric polymer having acid groups;
a film-forming aid; and
an alkaline material, wherein said alkaline material is present in an amount such that one or more carboxylic acid groups of the at least one enteric polymer has a degree of ionization of less than 15%.

103. The method of claim 102, wherein the alkaline material is at least one of ammonia, ethanolamine, diethanolamine, triethanolamine, lithium hydroxide, sodium hydroxide, potassium hydroxide, calcium hydroxide, magnesium hydroxide, sodium phosphate, sodium carbonate, sodium citrate, sodium ascorbate, lysine, arginine, or cationic polymers.

104. The method of claim 102, wherein the at least one enteric polymer is at least one of hydroxypropyl methylcellulose acetate succinate (HPMCAS), hydroxypropyl methylcellulose phthalate (HPMCP), cellulose acetate phthalate (CAP), acrylic polymers, or polyvinyl acetate phthalate (PVAP).

105. The method of claim 102, wherein the film-forming aid is at least one of hydroxypropyl methylcellulose (HPMC), methylcellulose (MC), gellan gum, or carrageenan.

106. The method of claim 100, wherein the initial dosage is at least 100 mg/day.

107.-109. (canceled)

110. The method of claim 100, wherein adjusting the initial dosage of the pharmaceutical composition comprises adjusting the initial dosage downward if the first patient exhibits an improvement as indicated by the second status.

111. The method of claim 110, wherein the initial dosage continues to be adjusted downward to an effective dose where the first patient still exhibits an improvement as measured by the indication of clinical efficacy.

112. The method of claim 100, wherein adjusting the initial dosage of the pharmaceutical composition comprises adjusting the initial dosage upward if the first patient does not exhibit an improvement as indicated by the second status.

113. The method of claim 112, wherein the initial dosage continues to be adjusted upward to an effective dose where the first patient exhibits an improvement as measured by the indication of clinical efficacy.

114. The method of claim 100, wherein an initial dosage of the pharmaceutical composition for a second patient is set at the adjusted dosage of the first patient if a baseline status of an indication of clinical efficacy of the second patient is similar to the second status of the indication of clinical efficacy of the first patient.

115. The method of claim 100, wherein adjusting the initial dosage of the pharmaceutical composition comprises adjusting the initial dosage downward if the first patient maintains an exceptional response in the timed walk distance and, concomitantly, a level of the at least one biomarker decreases significantly as compared to a previous level of the at least one biomarker.

116. The method of claim 100, wherein adjusting the initial dosage of the pharmaceutical composition comprises adjusting the initial dosage upward if the first patient no longer exhibits an exceptional response in the timed walk distance and, concomitantly, a level of the at least one biomarker is at least maintained relative to a previous level shown of the at least one biomarker.

117.-129. (canceled)

130. A method for treating a first patient with a pharmaceutical composition comprising imatinib or a pharmaceutically acceptable salt thereof, wherein the first patient is suffering from pulmonary arterial hypertension, the method comprising the steps of:
measuring an initial 6-minute walk distance of the first patient;
determining a circulating biomarker profile of the first patient, the circulating biomarker profile comprising a level or an expression pattern of at least one of a proteomic biomarker, a protein biomarker, a transcriptomic biomarker, a patient specific genetic signature, or an RNA expression biomarker signature prior to administration of the pharmaceutical composition;
administering the pharmaceutical composition to the first patient at an initial dosage of 240 mg for an initial treatment duration of 4 weeks and repeatedly adjusting the initial dosage by an increment of 120 mg/day for a treatment duration of 4 weeks until the first patient exhibits an improvement of greater than about 40 meters in the initial 6-minute walk distance, wherein the adjusted initial dosage that resulted in the improvement is an effective dosage;
classifying the first patient as a responder if the first patient exhibits an improvement;
determining the circulating biomarker profile after a treatment interval with the effective dosage; and
adjusting the effective dosage if the circulating biomarker profile is changed after the treatment interval.

131. The method of claim 130, further comprising, adjusting the effective dosage upward if the circulating biomarker profile indicates a decreased responsiveness of the responder.

132. The method of claim 130, further comprising, adjusting the effective dosage downward if the circulating biomarker profile indicates an increased responsiveness of the responder.

133. The method of claim 130, further comprising,
determining a circulating biomarker profile of a second patient and comparing it to the circulating biomarker profile of the first patient after the treatment interval; and
setting an initial dosage for the second patient to be an effective dose of the first patient if there is at least one similarity between the circulating biomarker profile of the second patient and the circulating biomarker profile of the first patient.

134. The method of claim 130, wherein the pharmaceutical composition is formulated for delayed release in a capsule.

135. The method of claim 134, wherein the capsule comprises:
at least one enteric polymer having acid groups;
a film-forming aid; and
an alkaline material.

136.-146. (canceled)
